(19) United States
(12) Patent Application Publication
(10) Pub. No.: US 2010/0016810 A1
(43) Pub. Date: Jan. 21, 2010

(54) DEVICES AND METHODS FOR FORMING TRACTS IN TISSUE

(75) Inventors: Michael DREWS, Palo Alto, CA (US); D. Bruce Modesitt, San Carlos, CA (US)

Correspondence Address: MORRISON & FOERSTER LLP, 755 PAGE MILL RD, PALO ALTO, CA, 94304-1018, US

(73) Assignee: Arstasis. Inc.,, San Carlos, CA (US)

(21) Appl. No.: 12/507,043

(22) Filed: Jul. 21, 2009

Related U.S. Application Data
(60) Provisional application No. 61/082,449, filed on Jul. 21, 2008.
(60) Provisional application No. 61/119,316, filed on Dec. 2, 2008.

(51) Int. Cl. A61M 5/32; A61B 17/32; A61B 1/32

(52) U.S. Cl. 604/272; 606/167; 600/201

(57) ABSTRACT

Described here are devices and methods for forming one or more tracts in tissue. The tracts may be formed in any suitable or desirable tissue, and may seal relatively quickly without the need for a supplemental closure device. In some variations, the methods may comprise clamping at least a portion of a tissue and advancing a tissue-piercing member through the clamped tissue to form a tract. The tract may, for example, provide access for one or more tools.

### CROSS-REFERENCE TO RELATED APPLICATIONS

This application claims the benefit of U.S. Provisional Application No. 61/082,449, filed Jul. 21, 2008, and U.S. Provisional Application No. 61/119,316, filed Dec. 2, 2008, the disclosures of both of which are incorporated herein by reference in their entirety.

### TECHNICAL FIELD

Described here are devices and methods for forming tracts in tissue. More specifically, described here are devices and methods for forming tracts in tissue where at least a portion of the tissue has been clamped or otherwise isolated.

### BACKGROUND

A number of devices and methods have previously been described for forming tracts in or through tissue. For example, devices and methods for forming tracts in tissue are described in U.S. patent application Ser. Nos. 10/844,247 (published as US 2005/0267520 A1), 10/888,682 (published as US 2006/0009802 A1), 11/432,982 (published as US 2006/0271078 A1), 11/544,149 (published as US 2007/0032802 A1), 11/544,177 (published as US 2007/0027454 A1), 11/544,196 (published as US 2007/0027455 A1), 11/544,317 (published as US 2007/0106246 A1), 11/544,365 (published as US 2007/0032803 A1), 11/545,272 (published as US 2007/0032804 A1), 11/788,509 (published as US 2007/0255313 A1), 11/873,957 (published as US 2009/0105744 A1), 12/467,251 (filed on May 15, 2009), and 61/178,895 (filed on May 15, 2009), all of which are incorporated herein by reference in their entirety. In general, the tracts described there may self-seal or seal without the need for a supplemental closure device. These tracts may be quite useful in providing access to a tissue location (e.g., an organ lumen) so that one or more tools may be advanced through the tract, and a procedure may be performed. Given the tremendous applicability of such methods, additional methods of forming tracts in tissue would be desirable.

### BRIEF SUMMARY

Described here are devices and methods for forming one or more tracts in tissue. Generally, tissue may be clamped or otherwise isolated, and positioned such that a tissue-piercing member may be used to form a tract in at least a portion of the tissue. In some variations, clamping or otherwise isolating the tissue may provide a general assessment as to the thickness of the tissue. The tracts may be formed in any suitable or desirable tissue. For example, the tissue may be an organ of any of the body systems (e.g., the cardiovascular system, the digestive system, the respiratory system, the excretory system, the reproductive system, the nervous system, etc.). In certain variations, the tissue is an organ of the cardiovascular system, such as the heart or an artery (e.g., the tract may be an arteriotomy). In other variations, the tissue is an organ of the digestive system, such as the stomach or intestines. The tracts formed here may seal relatively quickly without the need for a supplemental closure device. For example, the tracts may seal within 15 minutes or less, within 12 minutes or less, within 10 minutes or less, within 9 minutes or less, within 6 minutes or less, within 5 minutes or less, within 3 minutes or less, within 1 minute or less, etc. Of course, if necessary or desirable, one or more supplemental closure devices may be used in conjunction with the described devices and methods. In some variations, a single self-sealing tract may be formed in tissue, such as in a vessel wall. The single self-sealing tract may be formed, for example, by advancing only one tissue-piercing member through the tissue. This may, for example, result in minimal stress on the tissue.

The tissue-piercing member may be, for example, a needle, such as a hollow needle or a solid needle. The needle may have any suitable tip having any suitable shape (e.g., conical, offset conical, etc.). The tip may be blunt, sharpened or pointed, beveled or non-beveled, etc.

In some variations, a device may comprise a clamping member comprising first and second elongated clamping arms configured to clamp tissue therebetween, and a tissue-piercing member configured to form a tract in at least a portion of the clamped tissue. The device may further comprise a housing. One or both of the elongated clamping arms may be slidably disposed within, fixedly coupled to, or integral with the housing. The tissue-piercing member may be slidably disposed within the housing, the first elongated clamping arm, or any other suitable location. The housing may comprise at least one mechanism configured to operate the tissue-piercing member. The elongated clamping arms may be coupled to each other by a hinge. In certain variations, at least one of the elongated clamping arms may define at least one lumen therethrough, such as a guidewire lumen configured for allowing the clamping member to be advanced over a guidewire.

In some variations, a method may comprise clamping at least a portion of tissue of a subject between first and second elongated clamping arms of a clamping member, and advancing a tissue-piercing member through at least a portion of the clamped tissue to form a tract in the tissue. The method may further comprise advancing the clamping member through an opening in the tissue prior to clamping at least a portion of the tissue with the clamping member. The clamping member may, for example, be advanced over a guidewire. In certain variations, the tissue-piercing member may be advanced in multiple directions through at least a portion of the clamped tissue. In certain variations, the first elongated clamping arm may contact the portion of tissue, while the second elongated clamping arm does not contact the portion of tissue (e.g., while the second elongated clamping arm contacts a skin surface of the subject).

Methods described here may comprise manipulating at least a portion of the clamped tissue with the clamping member before, during, and/or after advancement of the tissue-piercing member. In some variations, the tissue may comprise a tissue wall, and manipulating at least a portion of the clamped tissue may comprise changing the orientation of the tissue wall from a first position to a second position and, in some cases, from a second position to a third position. In certain variations, the tissue-piercing member may be advanced through the manipulated tissue while the tissue wall is in the third position. In some variations, changing the tissue wall from one position to another position may comprise changing the shape of the tissue wall. Manipulating at least a portion of the clamped tissue may comprise changing the position of the tissue wall by rotating the tissue, tenting the tissue, etc. The method may further comprise immobilizing at least a portion of the clamped tissue.

The tissue-piercing member may enter the clamped tissue at a first location, and exit the clamped tissue at a second location, and the length between the first and second locations may be greater than the thickness of the tissue. In certain variations, the length of the tract may be greater than the thickness of the tissue. The method may further comprise advancing one or more closure devices and/or tools into and/or through the tract. In some variations, the tissue-piercing member may be advanced through at least a portion of the clamped tissue in an undulating fashion. The method may further comprise withdrawing the tissue-piercing member from the tissue. In certain variations, the tract may self-seal after the tissue-piercing member has been withdrawn from the tissue (e.g., within 15 minutes or less, 10 minutes or less, 5 minutes or less, 3 minutes or less, or 1 minute or less).

The tissue may be tissue of a vessel wall (e.g., an arterial wall). In some variations, the tissue may comprise an organ, such as an organ of the cardiovascular system, the digestive system (e.g., a stomach), the respiratory system, the excretory system, the reproductive system, or the nervous system. In certain variations, the organ may be an artery.

In some variations, a device may comprise an elongated member, a body and a foot portion coupled to the elongated member, and a tissue-piercing member configured to be advanced from the body, where the body is configured to displace one portion of a tissue in a first direction and the foot portion is configured to displace another portion of the tissue in a different direction (e.g., opposite the first direction). The foot portion may be articulatable. The device may further comprise a protrusion (e.g., a bump, ridge, lip, edge, etc.) on the body. In certain variations, the protrusion and the foot portion may be configured to clamp tissue therebetween. The protrusion may be used, for example, to provide an outer or upper reference, and/or to provide support for the tissue. In some variations, displacement of a portion of the tissue by the foot portion may position that portion of the tissue for piercing by the tissue-piercing member when the tissue-piercing member is advanced from the body. In certain variations, a method may comprise contacting a tissue with the device to displace one portion of the tissue in one direction and another portion of the tissue in a different direction, and advancing a tissue-piercing member through a displaced portion of the tissue to form a tract in the tissue.

In some variations, a device may comprise a clamping member comprising expandable regions configured to clamp tissue therebetween, and a tissue-piercing member configured to form a tract in at least a portion of the clamped tissue. In certain variations, the clamping member may further comprise an elongated member. In some variations, at least one of the expandable regions may be in the form of a region of the elongated member comprising at least one slit or opening. Alternatively or additionally, at least one of the expandable regions may comprise an inflatable member. In certain variations, a method may comprise clamping tissue between the expandable regions of the clamping member when at least two of the regions are in an expanded configuration, and advancing a tissue-piercing member through at least a portion of the clamped tissue to form a tract in the tissue.

In some variations, a device may comprise first and second curved surfaces that are opposed to each other and coupled at an attachment point, and that are configured to move about the attachment point between a first collapsed position and a second outwardly displaced position. The device may define a lumen configured to receive a tissue-piercing member, and the first and second curved surfaces, when in the first collapsed position, may be configured to clamp tissue and position at least a portion of the tissue for piercing by a tissue-piercing member passing through the lumen. The device may further comprise a tissue-piercing member (e.g., that is slidably disposed within a lumen of the device). The first and second curved surfaces, when in the first collapsed position, may be configured to substantially surround a vessel and position at least a portion of a wall of the vessel for piercing by a tissue-piercing member passing through the lumen. In certain variations, a method may comprise moving the first and second curved surfaces from the second outwardly displaced position to the first collapsed position to clamp tissue between the first and second curved surfaces, and to thereby position at least a portion of the clamped tissue for piercing by a tissue-piercing member passing through a lumen in the device. The method may further comprise advancing a tissue-piercing member through at least a portion of the clamped tissue.

In some variations, a method for forming a tract in tissue of a subject may comprise clamping at least a portion of tissue, and advancing a tissue-piercing member in a first direction through at least a portion of the clamped tissue to form a self-sealing tract in the tissue. Formation of the tract may require advancement of only one tissue-piercing member through the tissue. The method may also comprise advancing the tissue-piercing member in a second direction through at least a portion of the clamped tissue. The tissue may, for example, be clamped between first and second clamping portions of a clamping member, such as first and second elongated clamping arms. The clamping member may have a first position and a second position, and the first and second clamping portions may be farther apart from each other in the first position than they are in the second position. The method may comprise advancing the clamping member to the portion of tissue while the clamping member is in the second position. In some variations, the clamping member may be in the first position prior to clamping at least a portion of the tissue. In certain variations, the tissue may be clamped between opposed first and second curved surfaces coupled at an attachment point. The first and second curved surfaces may be configured to move about the attachment point between a first collapsed position and a second outwardly displaced position.

In some variations, a method for forming a tract in tissue of a subject may comprise clamping at least a portion of tissue, and advancing a tissue-piercing member in a first direction through at least a portion of the clamped tissue to form a single tract in the tissue, where the single tract is self-sealing. In certain variations, a method for forming a tract in tissue of a subject may comprise using a device to clamp at least a portion of tissue, and forming a tract in the tissue by advancing at least one tissue-piercing member through at least a portion of the clamped tissue, where formation of the tract requires advancement only of the tissue-piercing member through the tissue, and where the tract is self-sealing.

### BRIEF DESCRIPTION OF THE DRAWINGS

FIGS. 1A-1N depict variations of a method and a device for forming a tract in tissue.

FIGS. 2A-2C show a variation of a device for forming a tract in tissue.

FIGS. 3A-3I illustrate a variation of a method for forming a tract in tissue using the device shown in FIGS. 2A-2C.

FIGS. 4A-4E are side views of variations of components of devices for forming a tract in tissue.

FIGS. 5A and 5B are side views of a variation of a device for forming a tract in tissue.

FIGS. 6A-6D show side views of additional variations of devices for forming a tract in tissue.

FIG. 7 illustrates variations of a method and a device for forming a tract in tissue.

FIGS. 8A and 8B are side views of another variation of a device for forming a tract in tissue.

FIGS. 9A and 9B are front views of additional variations of devices for forming a tract in tissue.

FIGS. 10A and 10B are side views of a further variation of a device for forming a tract in tissue.

FIGS. 11A-11D illustrate variations of a method and a device for forming a tract in tissue.

FIGS. 12A-12C show variations of devices for forming a tract in tissue.

FIGS. 13A-13E depict variations of a method and a device for forming a tract in tissue.

FIGS. 14A and 14B show variations of a method and a device for forming a tract in tissue.

FIGS. 15A-15C show additional variations of a method and a device for forming a tract in tissue.

FIGS. 16A and 16B show variations of a method and a device for forming a tract in tissue.

FIGS. 17A and 17B show further variations of a method and a device for forming a tract in tissue.

FIGS. 18A-18C show additional variations of a method and a device for forming a tract in tissue.

FIGS. 19A and 19B show variations of a method and a device for forming a tract in tissue.

FIGS. 20A and 20B show further variations of a method and a device for forming a tract in tissue.

FIGS. 21A and 21B show additional variations of a method and a device for forming a tract in tissue.

FIGS. 22A and 22B depict variations of a device and a method for forming a tract in tissue.

FIG. 23A is a perspective view of a variation of a device for forming a tract in tissue, when a clamping member of the device is withdrawn; FIG. 23B is a side view of the device of FIG. 23A; and FIG. 23C is a front view of the device of FIG. 23A.

FIG. 23D is a perspective view of the device of FIGS. 23A-23C, when the clamping member of the device is deployed; FIG. 23E is a side view of the device of FIG. 23D; and FIG. 23F is a front view of the device of FIG. 23D.

FIGS. 24A-24C are perspective, side, and front views, respectively, of the device of FIGS. 23A-23F during a first stage of deployment of the clamping member; FIGS. 24D-24F are perspective, side, and front views, respectively, of the device of FIGS. 23A-23F during a second stage of deployment of the clamping member; FIGS. 24G-24I are perspective, side, and front views, respectively, of the device of FIGS. 23A-23F during a third stage of deployment of the clamping member; and FIGS. 24J-24L are perspective, side, and front views, respectively, of the device of FIGS. 23A-23F during a fourth stage of deployment of the clamping member.

FIGS. 25A and 25B are perspective and side views, respectively, of the device of FIGS. 23A-23F, illustrating a path of advancement of a tissue-piercing member from the device into a vessel wall, and FIG. 25C is a cross-sectional view taken along line 25C-25C of FIG. 25B.

FIG. 26 is a schematic illustration of variations of a device and a method for forming a tract in tissue.

### DETAILED DESCRIPTION

Described here are devices and methods for forming tracts in tissue. In general, tracts formed by the devices and methods described here may seal relatively quickly, without the need for a supplemental closure device. In some variations, the devices comprise one or more clamping members, expandable regions, and/or other components that may be used to isolate, immobilize, and/or position tissue for tract formation. This may allow for relatively accurate, easy, and efficient tract formation. In certain variations, the devices and/or methods described here may be used to position a tissue-piercing member at a specific location in a portion of tissue such that the tissue-piercing member can form a tract in the specific location. In some variations, devices and/or methods described here may be used to form a single tract in tissue, where the single tract is self-sealing. The tract may self-seal relatively quickly after a procedure, and may thereby result in reduced procedure time.

It should be understood that the devices and methods described here may be used with any tissue in which it is desired to form one or more tracts. For example, the tissue may be an organ, such as an organ of any of the body systems (e.g., the cardiovascular system, the respiratory system, the excretory system, the digestive system, the reproductive system, the nervous system, etc.). In some variations, the tissue is an organ of the digestive system, such as the stomach, or intestines. In other variations, the methods are used with tissue of the cardiovascular system, such as the vasculature or the heart. As an example, one or more tracts may be formed through a muscular wall and/or septum of a heart to access the left ventricle, the aorta, the aortic valve, the mitral valve, the aortic arch, etc. For example, a tissue-piercing member may be used to form a tract from a peripheral surface of a heart, through a muscular wall of the heart, and into a septum of the heart. In certain variations, a tissue-piercing member may be used to form a transapical tract in a heart. In some variations, the tissue is an artery, and the methods are used in conjunction with performing an arterial puncture.

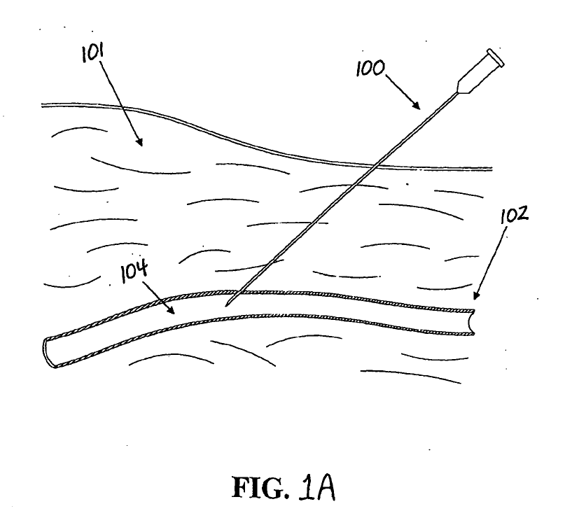

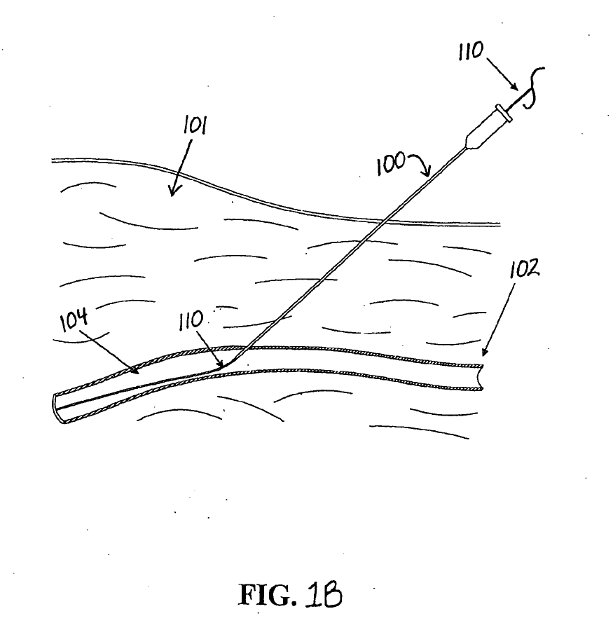

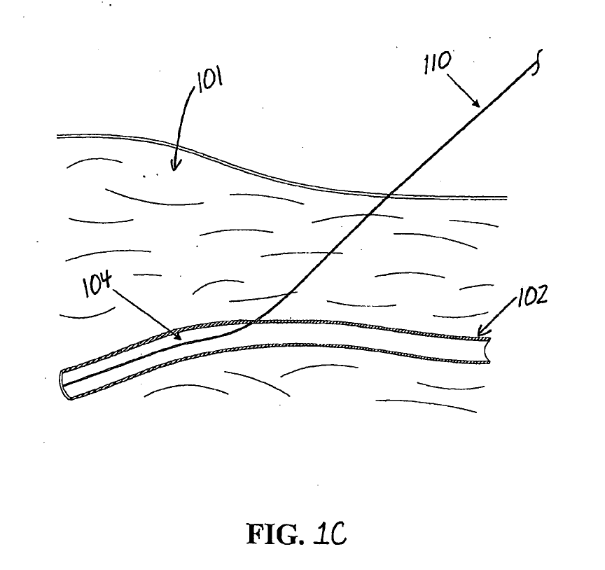

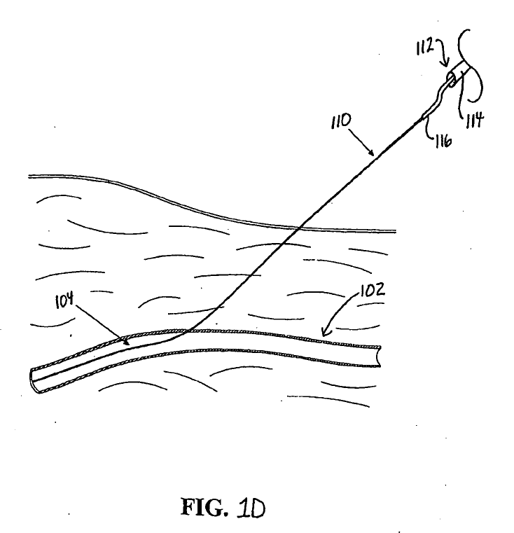

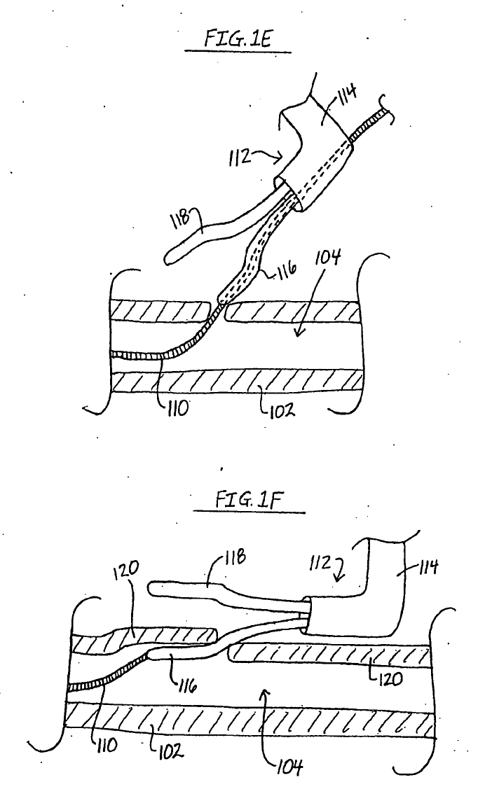

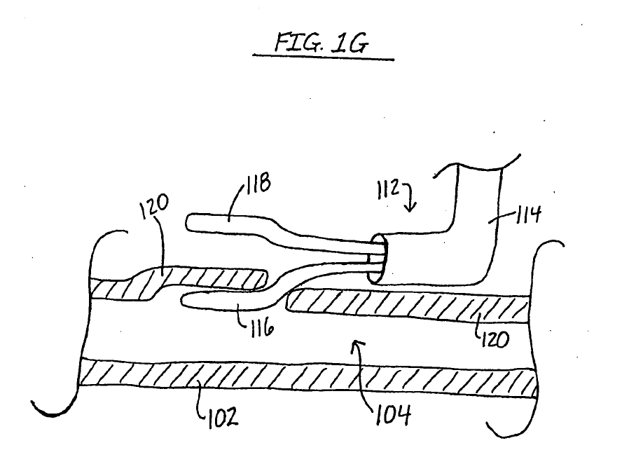

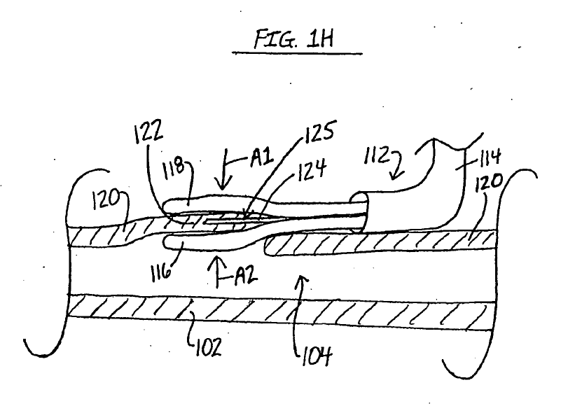

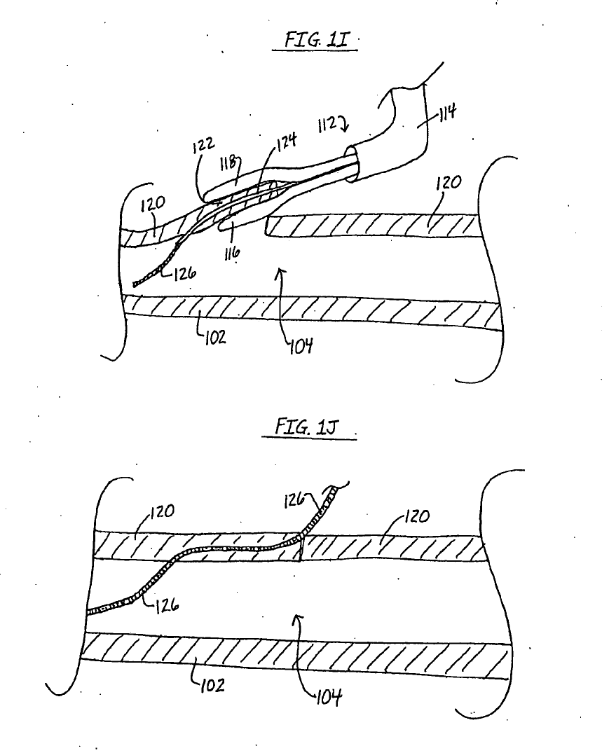

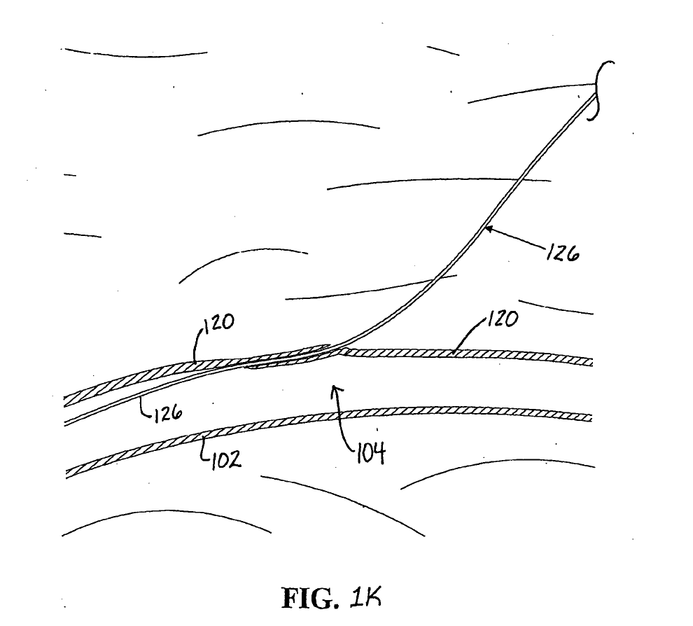

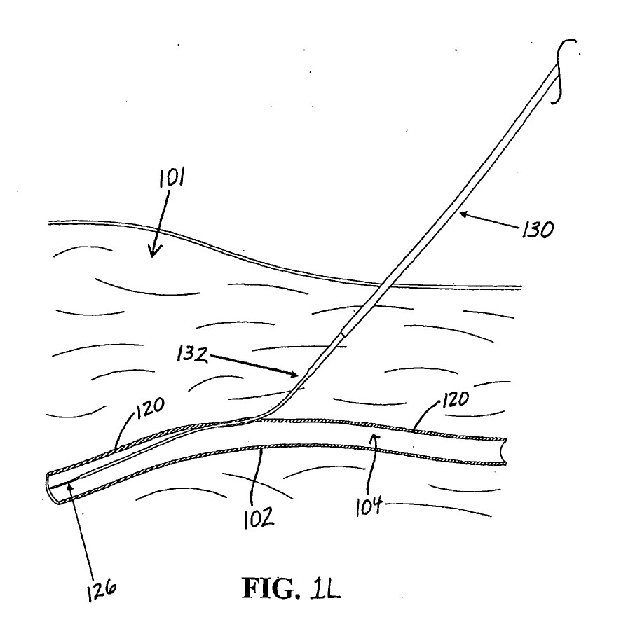

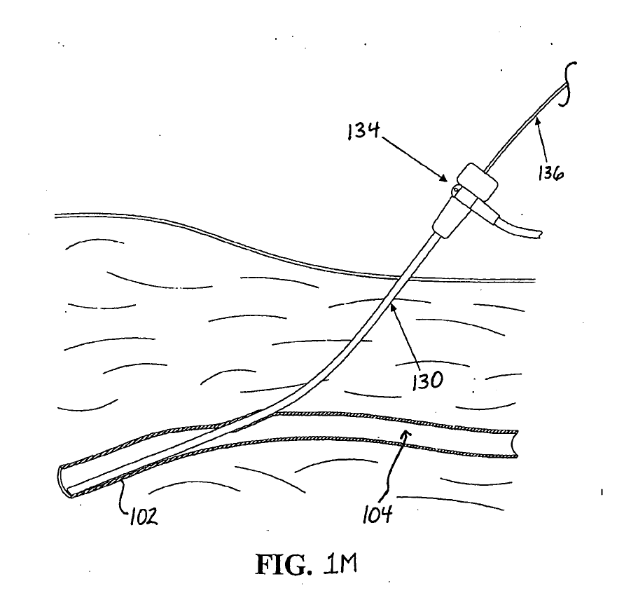

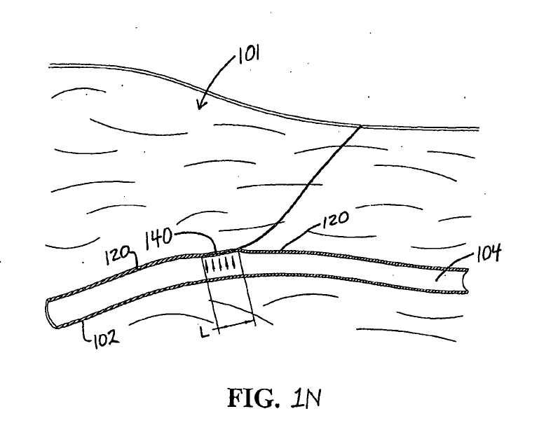

Turning now to FIGS. 1A-1N, variations of a device and a method for forming one or more tracts in tissue are illustrated. While FIGS. 1A-1N show the formation of a tract in arterial tissue, it should be understood that the devices and methods described here may be used with any suitable tissue, as described above.

FIGS. 1A-1C show a standard Seldinger procedure for placement of a wire through a tissue. First, and as shown in FIG. 1A, a needle (100) is advanced through subcutaneous tissue (101) into an artery (102). As shown, needle (100) has entered a lumen (104) of artery (102). Entry into lumen (104) by needle (100) may optionally be visually confirmed by observing a flash of blood (i.e., blood flow) through the needle. FIG. 1B shows advancement of a wire (110) through needle (100) and into lumen (104) of artery (102). After placement of wire (110), the needle may be withdrawn proximally, leaving wire (110) in lumen (104), as shown in FIG. 1C.

FIG. 1D shows a variation of a device (112) that may be used to form one or more tracts in tissue (e.g., in accordance with the various methods described here). As shown in FIG. 1D, device (112) comprises a housing (114) and a clamping arm (116) fixedly coupled to housing (114). Clamping arm (116) defines a lumen therethrough (not shown) that allows clamping arm (116) to be advanced over wire (110), as depicted in FIG. 1D. Device (112) also includes a retractable second clamping arm, shown in FIG. 1E and discussed in further detail below. Any suitable material or materials may be used for device (112). For example, the device may comprise one or more biocompatible plastic materials (e.g., an injection molded polycarbonate), stainless steels, shape memory materials, combinations thereof, or any other suitable materials.

In FIG. 1D, device (112) is being advanced over wire (110), toward artery (102). As device (112) is being advanced toward artery (102), the second clamping arm of device (112) may be retracted into housing (114) of device (112). This may allow device (112) to maintain a relatively low profile which may, in turn, allow device (112) to be delivered to a target site relatively easily.

Referring now to FIG. 1E, and as discussed above, device (112) further includes a retractable clamping arm (118) that is slidably disposed relative to housing (114). Clamping arm (118) may be withdrawn or retracted into housing (114), or deployed from housing (114), as desired. As shown in FIG. 1E, because device (112) is nearing the target site (i.e., artery (102)), clamping arm (118) has been deployed from housing (114). However, other variations of methods may comprise deploying a second clamping arm at an earlier or later time. In some variations, device (112) may include a handle portion having an actuation mechanism, such as a button or a slide mechanism, that is connected to clamping arm (118). The mechanism may be used to slide clamping arm (118) into and out of housing (114).

Referring now to FIGS. 1F-1H, device (112) may be used to clamp tissue so that the tissue is isolated and/or positioned for a tissue-piercing member to form a pre-defined tract through the tissue. As shown in FIG. 1F, clamping arm (116) is advanced over wire (110), through a wall portion (120) of artery (102), and into lumen (104) of artery (102). In certain variations, device (112) may comprise a port (not shown) such that once device (112) has been advanced into a vessel lumen, a flash of blood is released from the port. For example, the port may be in fluid communication with clamping arm (116), so that when clamping arm (116) reaches lumen (104) of artery (102), blood flows into clamping arm (116) and toward the port, exiting the port as a flash of blood. This flash of blood may be used as a signal that ensures that clamping arm (116) has been advanced so that it fully resides within lumen (104) of artery (102). While clamping arm (116) is disposed within lumen (104) of artery (102), clamping arm (118) remains external to artery (102). In some variations, device (112) (e.g., clamping arm (116)) may be rotated during insertion into a target site (such as artery (102)). For example, device (112) may be rotated 45 degrees, 90 degrees total, etc. Of course, any degree of rotation, in either direction, may be used as desirable.

In certain variations, device (112) may further comprise a retainer that may be used to help position clamping arm (116) within lumen (104) of artery (102). The retainer may, for example, be in the form of a foot or a loop, or any other suitable shape. In some variations, the retainer may be deployed from a retainer opening in clamping arm (116). In certain variations, a latch on device (112) may be used to maintain the retainer in the retainer opening in its undeployed position when desirable. Certain of Applicant's previous applications incorporated by reference above disclose using a retainer to aid in positioning a tissue-locating member. Here, a retainer would similarly be used to position clamping arm (116).

After clamping arm (116) has been positioned within lumen (104), wire (110) may be proximally withdrawn (FIG. 1G). Referring now to FIG. 1H, clamping arm (118) may then be moved in the direction of arrow (A1), while clamping arm (116) is moved in the direction of arrow (A2). As a result, the two clamping arms clamp tissue (122) of wall portion (120) between them. This clamping action may be effected, for example, using an actuation mechanism in device (112). As also shown in FIG. 1H, after tissue (122) has been clamped, a tissue-piercing member (124) (as shown, a needle, although other suitable tissue-piercing members may be used) is advanced from device (112) (e.g., through one or more channels, ports, and/or openings in device (112)) such that it pierces the clamped portion of tissue (122). FIG. 1H shows tissue-piercing member (124) advancing laterally into clamped portion of tissue (122) at a first location (125), although a tissue-piercing member may enter a portion of tissue at any suitable angle relative to the tissue. In some variations, a tissue-piercing member may enter a tissue portion at a compound angle relative to the tissue portion. As an example, a tissue-piercing member may enter a vessel lumen at a compound angle relative to the vessel's longitudinal axis.

Referring to FIG. 1I, the clamped tissue may be manipulated (e.g., rotated, tented, etc.) as tissue-piercing member (124) is advanced through it, until tissue piercing member (124) enters lumen (104). At that point, the tissue-piercing member has formed a tract through tissue (122). Manipulation of the tissue during tract formation may cause the tissue-piercing member to change direction as it is advanced through the tissue. However, it should be noted that while tissue manipulation has been described, some variations of methods may include little or no tissue manipulation.

In certain variations, as tissue-piercing member (124) is advanced into lumen (104) of artery (102), a flash of blood may be visualized (e.g., through a marker port on device (112)). In this way, proper positioning of tissue-piercing member (124) within the lumen may be confirmed. If advancement of tissue-piercing member (124) does not result in entry in the lumen (e.g., if calcification prevents proper needle redirection, or if there is unfavorable anatomy or device positioning, etc.), then device (112) may be withdrawn proximally, and a decision may be made to try the procedure with another device, or to use a standard arteriotomy procedure (in the case where the tissue is an artery).

As shown in FIG. 1I, tissue-piercing member (124) may be hollow, and/or may comprise one or more lumens and/or apertures, so that a wire (126) can be advanced through tissue-piercing member (124) and into lumen (104). Of course, while a hollow tissue-piercing member has been described, in certain variations, a solid tissue-piercing member may be used. Moreover, some variations of devices may comprise more than one tissue-piercing member. As an example, a device may comprise a first tissue-piercing member having a relatively large cross-sectional diameter, and a second tissue-piercing member having a relatively small cross-sectional diameter. The relatively large tissue-piercing member may be selected, for example, to form a tract for deployment of a relatively large tool to a target site, while the relatively small tissue-piercing member may be selected, for example, to form a tract for deployment of a relatively small tool to a target site. Such selectivity may allow one access device to be used for many different procedures. As another example, in certain embodiments, a device may comprise a tissue-piercing member in the form of a needle within a needle.

Referring again to FIG. 1I, wire (126) may be the same wire as wire (110) described above, or may be a different wire. After wire (126) has been advanced through the tissue-piercing member and into lumen (104), wire (126) may then act as a guide for advancement of one or more tools into the lumen. For example, and as shown in FIG. 1J, device (112) may be proximally withdrawn from artery (102), leaving wire (126) behind. FIG. 1K shows an expanded view of wire (126) crossing into artery (102). Once wire (126) has been placed in lumen (104), wire (126) may be used to position one or more devices and/or tools in lumen (104). For example, FIG. 1L shows advancement of a sheath (130) over wire (126) for introduction of one or more tools therethrough.

As shown in FIG. 1L, sheath (130) is slidably coupled to a dilator (132). The dilator may be advanced through the lumen of sheath (130), and be used to facilitate positioning of the sheath in lumen (104) of artery (102) (or other tissue as the case may be). As shown in FIG. 1L, dilator (132) has an elongated tip, having a distal cross-sectional diameter that is smaller than the cross-sectional diameter near its proximal end. This type of sheath/dilator system may be particularly advantageous if sheath (130) has a much greater cross-sectional diameter (e.g., 5F-12F) than the wire (e.g., 0.012 to 0.35 inch) over which it will be advanced, since the wire may not provide sufficient structural support for insertion of the sheath. Here, the end of dilator (132) having a smaller cross-sectional diameter is more easily advanced over wire (126) and thus provides better support for the larger diameter portions to follow. In this way, the cross-sectional area of the tract is gradually increased, which may help in reducing trauma to the tissue. FIG. 1M shows sheath (130) in lumen (104) after dilator (132) has been withdrawn. Also shown there is the proximal end (134) of the sheath having an opening therein for introduction of one or more tools (136).

FIG. 1N shows the tract (140) formed in the tissue, after the device and any additional tools have been withdrawn. If desirable, a filament (e.g., a wire, a polymer, etc.) or suture material having any suitable cross-section may be left in the tract and exit the body. In this way, if re-access to the lumen is desirable (for example, for placement of a supplemental closure device, for performing additional procedures, etc.), the filament or suture may be used as a guide over which re-access may be accomplished using one or more tools.

Tract (140) is generally diagonal, and has a length (L). The length of the tract may be any suitable or desirable length to help facilitate relatively rapid sealing of the tract. For example, when the devices and methods described here are used with the vasculature, a longer tract may be desirable, as it is believed that a longer tract may expose helpful biological factors (e.g., growth factors, etc.) that may aid in sealing the tract. This may also be the case with other tissue as well. In addition, a longer tract will have a larger area for mechanical pressure to act on, which may cause the tract to seal more quickly. In some variations, length (L) is greater than the thickness of wall portion (120) (e.g., in the location of wall portion (120) where tract (140) is formed). The arrows shown in FIG. 1N illustrate how pressure acting on the tract causes the tract to seal relatively rapidly without the need for an additional closure device. For example, the tract may seal in 12 minutes or less, 9 minutes or less, 6 minutes or less, 3 minutes or less, etc., reducing the duration of any external compression that may be needed. Of course, if desirable, an additional closure device (e.g., plug, clip, glue, suture, etc.) may be used.

While device (112) is shown as including one clamping arm that is fixedly coupled to its housing and another clamping arm that is slidably disposed relative to its housing, other variations of devices may comprise different combinations and/or arrangements of clamping arms. As an example, one variation of a device may comprise a housing and multiple clamping arms that are fixedly coupled to the housing. As another example, another variation of a device may comprise a housing and multiple clamping arms that are slidably disposed relative to the housing. Some variations of devices may not include any fixed clamping arms, and certain variations of devices may not include any slidable clamping arms. Any suitable combination of fixed and slidable clamping arms may also be used in a device. Devices may comprise any suitable number of clamping arms (e.g., three, four, five, ten, etc.), which can be fixedly coupled, slidably disposed, or a combination thereof. Moreover, some variations of devices may comprise only one clamping arm (e.g., that is configured to interact with another component of the device to clamp tissue). Additionally, certain variations of devices may comprise one or more clamping arms without also comprising a housing. For example, a device may comprise two clamping arms that are connected to each other via a hinge. Examples of hinged devices are described in further detail below.

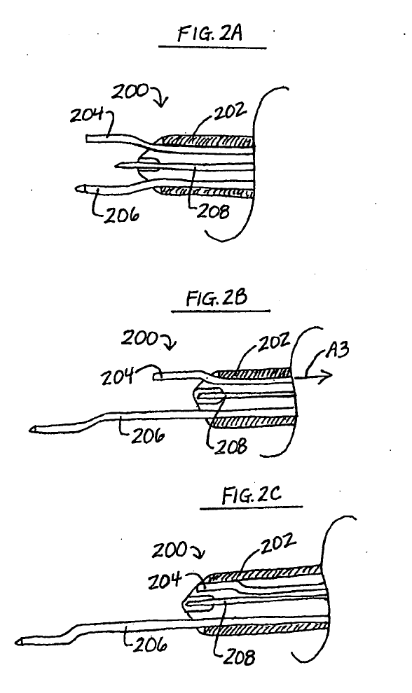

FIGS. 2A-2C show another variation of a clamping device that may be used to form a tract in tissue. As shown there, a device (200) comprises a housing (202), as well as two clamping arms (204) and (206) and a tissue-piercing member (208) partially disposed within the housing. FIG. 2A shows device (200) when both of the clamping arms and the tissue-piercing member partially extend from housing (202). However, and as shown in FIGS. 2B and 2C, clamping arm (204) and tissue-piercing member (208) may be retracted into housing (202), by moving them in the direction of arrow (A3). Such retraction may occur, for example, prior to delivery of device (200) to a target site. Furthermore, while not shown, in some variations both clamping arms (204) and (206) may be capable of being retracted into housing (202). The retraction of one or both of the clamping arms into the housing may make it easier to deliver device (200) to a target site (e.g., by decreasing the profile of device (200) and making it less likely that any of the components of device (200) will catch on tissue during delivery). As shown in FIGS. 2B and 2C, in certain variations, clamping arm (206) can be distally extended from housing (202) (e.g., when it is desired to clamp tissue that is relatively far away from device (200)). While not shown, in some variations, clamping arm (204) can alternatively or additionally be distally extended from housing (202).

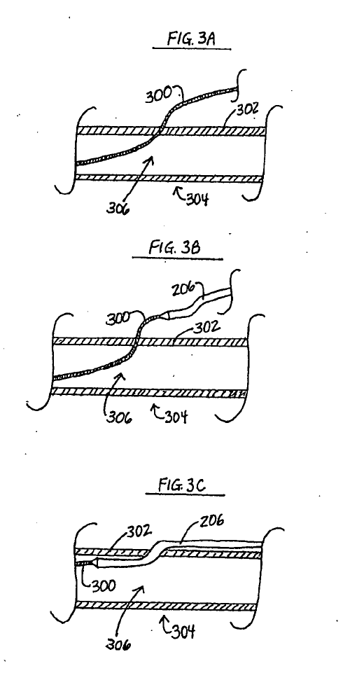

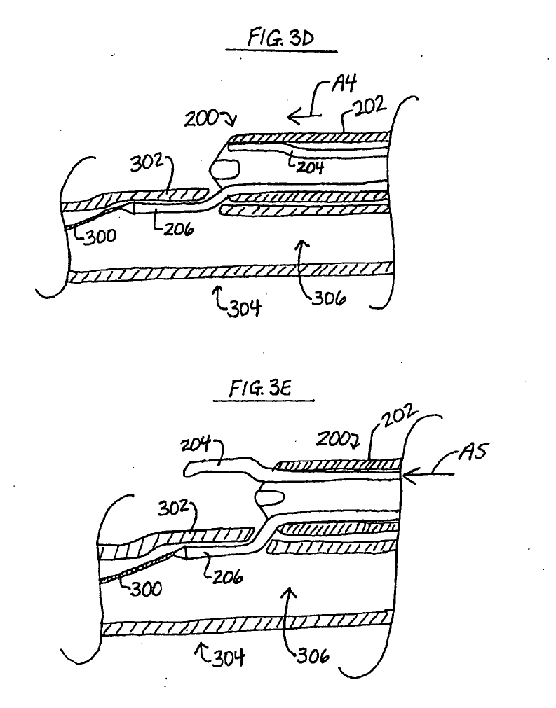

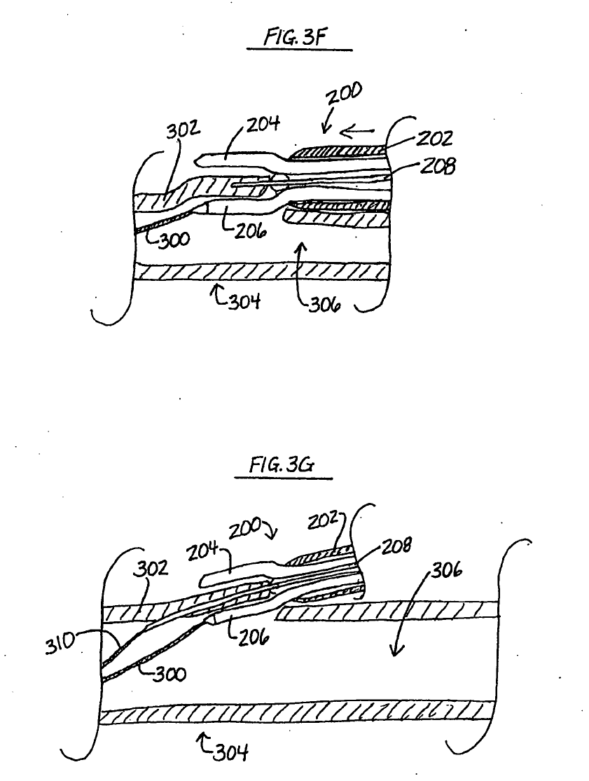

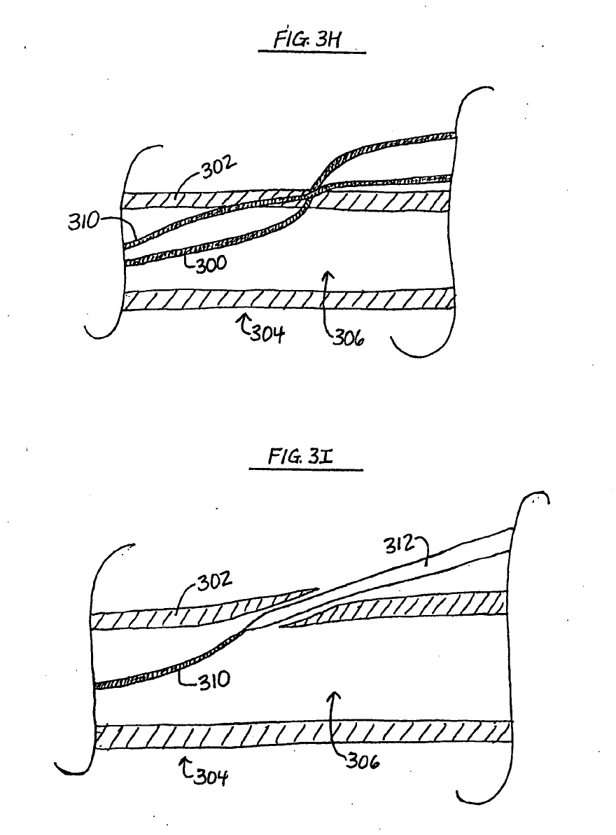

FIGS. 3A-3I illustrate a method of using clamping device (200) to form a tract in a portion of tissue. First, and referring to FIG. 3A, a wire (300) is delivered through a wall portion (302) of a tissue portion (304), and into a hollow region (306) of the tissue portion. Referring now to FIGS. 3B and 3C, clamping arm (206) of device (200) may then be delivered over wire (300) and into hollow region (306). Next, and as shown in FIG. 3D, housing (202) may be advanced (e.g., pushed) distally (in the direction of arrow (A4)), such that a portion of clamping arm (206) goes into housing (202). Clamping arm (204) may then be deployed from housing (202) (FIG. 3E), in the direction of arrow (A5) and may be used to clamp wall portion (302) of tissue portion (304), as shown in FIG. 3F. Next, and as also shown in FIG. 3F, tissue-piercing member (208) may be deployed from housing (202) and into wall portion (302) of tissue portion (304).

Clamping arms (204) and (206) are configured to clamp tissue, such as tissue portion (304), in such a way that tissue-piercing member (208) enters the tissue at a pre-selected or desired location. The distal advancement of housing (202) prior to deployment of the tissue-piercing member (FIG. 3D) may further help the clamping arms to achieve this positioning. However, in some variations, the housing may not be distally advanced. As shown in FIG. 3F, clamping arms (204) and (206) clamp wall portion (302) and secure it such that tissue-piercing member (208) pierces through the center of the wall portion. Thus, clamping device (200) may provide relatively predictable deployment and advancement of tissue-piercing member (208) through tissue. While FIG. 3F shows tissue-piercing member (208) entering the center of wall portion (302), in some variations, a tissue-piercing member may be advanced from a clamping device such that the tissue-piercing member enters a clamped portion of tissue off-center.

Referring now to FIG. 3G, tissue-piercing member (208) is advanced through wall portion (302) until the tissue-piercing member enters hollow region (306). Thereafter, a wire (310) may be advanced through tissue-piercing member (208), such that wire (310) crosses wall portion (302) and enters hollow region (306) of tissue portion (304). Device (200) (including tissue-piercing member (208)) may then be proximally withdrawn, leaving wires (300) and (310) behind, as shown in FIG. 3H. Finally, wire (300) may be withdrawn from tissue portion (304) and, as shown in FIG. 3I, a sheath or tool (312) may be advanced over wire (310) and into tissue portion (304). One or more procedures may then be performed as desired.

The clamping arms shown above are depicted as having generally uniform cross-sectional diameters. However, in some variations, a clamping arm may have a non-uniform cross-sectional diameter. For example, a distal portion of the clamping arm may have a relatively large cross-sectional diameter, while a proximal portion of the clamping arm has a relatively small cross-sectional diameter. In certain variations, the cross-sectional diameter of a clamping arm may gradually increase along the length of the clamping arm. Moreover, in some variations, a clamping arm may have a non-circular cross-sectional shape, such as a square or hexagonal cross-sectional shape.

While the clamping arms shown above are depicted as having generally rounded and smooth surfaces, any suitable configuration of clamping arm may be used in the devices and methods described herein. For example, one or more clamping arms of a device may have at least one grooved surface, serrated surface, porous surface, spiked surface, abrasive surface, etc. Combinations of different types of surfaces may also be used (e.g., a clamping arm may have a portion with a grooved surface and a portion with a spiked surface, or a portion with a serrated surface and a portion with a smooth surface, etc.). Certain types of surfaces (e.g., a serrated surface) may enhance the grip of a clamping arm. In some variations, a single device may include multiple (e.g., two, three, four, five) clamping arms, where at least two of the clamping arms have different surfaces.

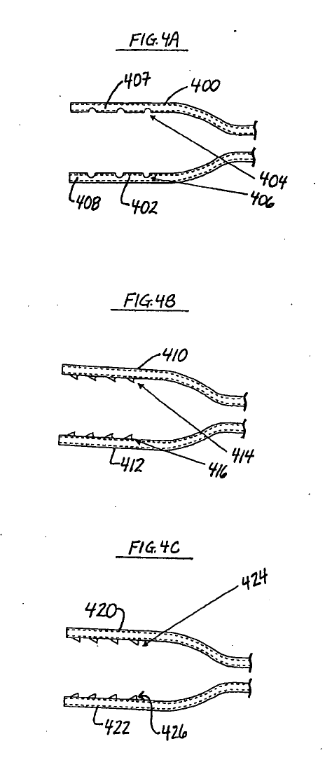

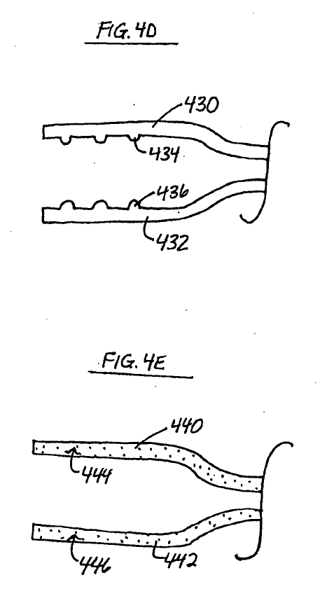

Examples of different variations of clamping arms are shown in FIGS. 4A-4E. FIG. 4A shows clamping arms (400) and (402) having pitted regions (404) and (406), respectively. In some instances, tissue may fold or invaginate into the pitted regions when clamping arms (400) and (402) are used to clamp the tissue. This may, for example, enhance tissue traction and clamping security. As shown, clamping arm (400) has a lumen (407) and clamping arm (402) has a lumen (408). A clamping arm lumen may be used, for example, to advance a clamping arm over a wire and/or to deliver one or more therapeutic agents through the clamping arm to a target site. The lumen may also be used for any other suitable purpose. For example, the lumen may be connected to a vacuum source to provide a vacuum that aids in tissue-gripping. While clamping arms (400) and (402) are each depicted as having one lumen, some variations of clamping arms may include multiple lumens (e.g., two lumens, three lumens). For example, one lumen maybe used to advance a clamping arm over a wire, while another lumen may be used to deliver one or more therapeutic agents from the clamping arm. Moreover, some variations of clamping arms may not include any lumens.

Referring back to the figures, FIG. 4B shows clamping arms (410) and (412) having spikes (414) and (416), respectively. The spikes may help clamping arms (410) and (412) to better engage tissue. Spikes (414) and (416) are depicted as being somewhat fin-shaped, and as including curved sections. However, in some variations, more angular spikes may be employed. As an example, FIG. 4C shows clamping arms (420) and (422) having angular spikes (424) and (426), respectively. Rounded protrusions may alternatively or additionally be used. For example, FIG. 4D shows clamping arms (430) and (432) including rounded protrusions (434) and (436), respectively. In certain variations, a clamping arm may have a textured or otherwise modified surface. As an example, FIG. 4E shows clamping arms (440) and (442) having roughened surfaces (444) and (446), respectively. The roughened surfaces may, for example, enhance the grip of the clamping arms on tissue.

In some variations, a device may include at least one clamping arm comprising one or more coatings, such as a polymer coating. The coating or coatings may, for example, provide enhanced gripping of a tissue surface. As an example, in some variations, a clamping arm may comprise a silicone coating. In certain variations, a clamping arm may comprise one or more hydrophilic coatings and/or one or more hydrophobic coatings. As an example, one portion of a clamping arm may be coated with a hydrophilic coating, while another portion of the clamping arm is coated with a hydrophobic coating. In some variations, the type of coating that is used on at least a portion of a clamping arm may be selected based on the type of tissue to be clamped by the clamping arm.

Clamping arms such as those shown in FIGS. 4A-4E may be formed, for example, by modifying (e.g., cutting, abrading, bead blasting, etc.) hypotubes, or may be formed using any other suitable method. In some variations, clamping arms may be formed by applying one or more surface treatments and/or rough coatings to hypotubes. The clamping arms shown in FIGS. 4A-4E are only examples of different possible clamping arms that may be used, and other variations and combinations of clamping arms (e.g., clamping arms comprising ridges, hooks, barbs, etc.) may be used as appropriate.

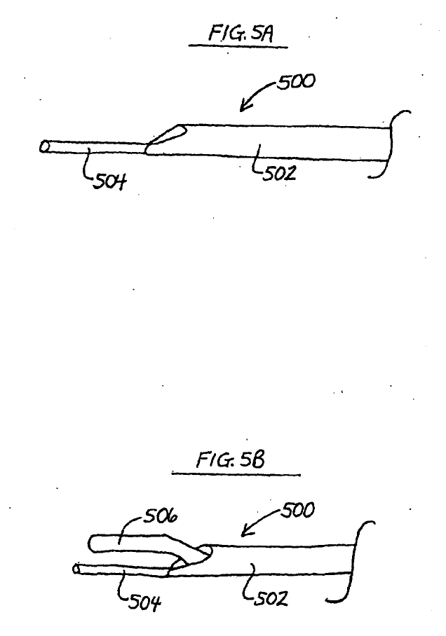

As an example, FIGS. 5A and 5B show a variation of a device (500) that may be used to clamp tissue (and, e.g., form one or more tracts in the clamped tissue). As shown there, device (500) comprises a housing (502) and a clamping arm (504) fixedly coupled (e.g., press-fitted, snap-fitted, sonically welded, laser-welded, soldered, brazed, glazed, fastened, etc.) to housing (502). Referring specifically now to FIG. 5B, device (500) further includes a second clamping arm (506) that is slidably disposed within housing (502), and that is capable of being advanced from housing (502) or retracted into housing (502) as desired. As shown, second clamping arm (506) may be offset when advanced from housing (502), such that it increases the overall profile of device (500) when advanced. However, the retractability of second clamping arm (506) into housing (502) may allow device (500) to maintain a relatively low profile during delivery to a target site. It should be noted that other sizes and configurations of clamping arms may also be used, as appropriate. For example, a device may not include any clamping arms that are offset when advanced from a housing of the device, or may include one or more clamping arms that extend within the profile of a housing of the device when advanced from the housing.

Device (500) may be particularly well-suited for use in, for example, remote access procedures, minimally invasive procedures, and/or mini-incision procedures. While clamping arm (504) is relatively cylindrical in shape, clamping arm (506) is curved (e.g., to provide enhanced tissue engagement). Additionally, clamping arm (506) has a larger cross-sectional diameter than clamping arm (504).

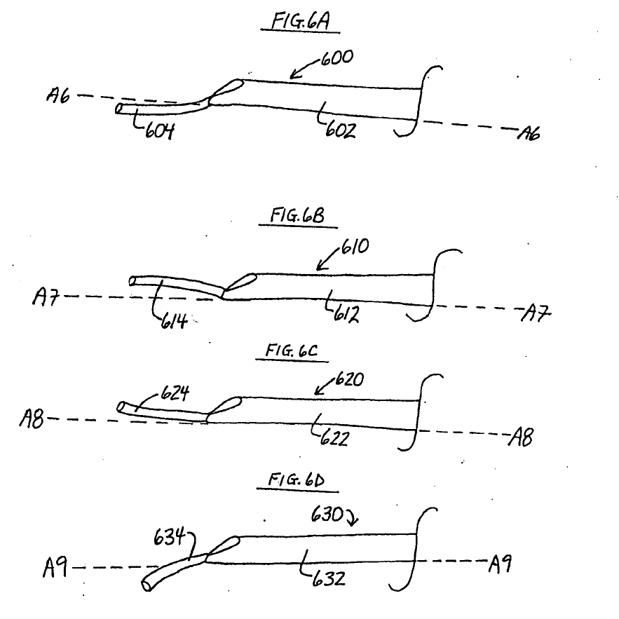

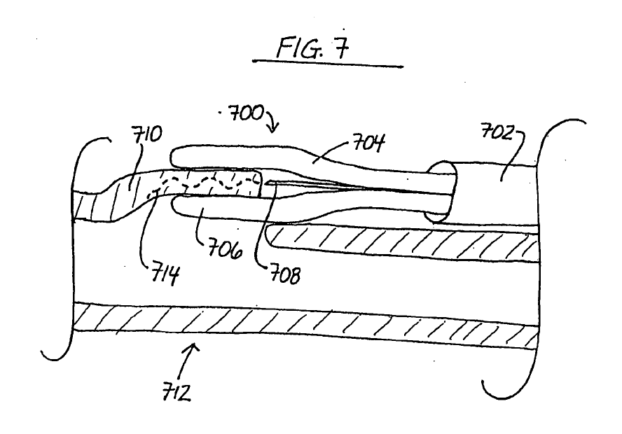

Of course, other combinations of sizes and shapes of clamping arms may be used. For example, FIG. 6A shows a device (600) comprising a housing (602) and a clamping arm (604) that is fixedly coupled to the housing and that forms a concave curve extending downward relative to a bottom axis (A6) of the housing. FIG. 6B shows a device (610) comprising a housing (612) and a clamping arm (614) that is fixedly coupled to the housing and that forms a convex curve extending upward relative to a bottom axis (A7) of the housing. FIG. 6C shows a device (620) comprising a housing (622) and a clamping arm (624) that is fixedly coupled to the housing and that forms a concave curve extending upward relative to a bottom axis (A8) of the housing. Finally, FIG. 6D shows a device (630) comprising a housing (632) and a clamping arm (634) that is fixedly coupled to the housing and that forms a convex curve extending downward relative to a bottom axis (A9) of the housing. The different configurations of the clamping arms may be appropriate for use, for example, with different tissue types.

While the above tissue-clamping devices show tissue-piercing members taking lateral paths and/or curved paths through tissue, tissue-piercing members may be configured to take any of a number of different kinds of paths through tissue, as appropriate. For example, FIG. 7 shows a device (700) comprising a housing (702), two clamping arms (704) and (706) extending from the housing, and a tissue-piercing member (708) that may be deployed from the housing between the two clamping arms. As shown there, the tissue-piercing member is clamping a wall portion (710) of an artery (712). Tissue-piercing member (708) is configured to advance through wall portion (710) along an undulating path, and may thereby form an undulating tract through the wall portion tissue. Any of a number of different mechanisms may be used to form an undulating tract. As an example, in some variations, the tissue-piercing member may be advanced through an exit port in the housing that is capable of being articulated, tilted, and/or flexed, and that may thereby cause the tissue-piercing member to undulate as it is being advanced. As another example, in certain variations, a cam follower system may be employed to cause the tissue-piercing member to form an undulating tract when deployed from the housing. Alternatively or additionally, the proximal end of the tissue-piercing member may be manipulated (e.g., pushed, driven, etc.) to cause the tissue-piercing member to form an undulating tract. The undulating tract may, for example, have a greater surface area than tracts formed by other tissue-piercing members that follow a relatively straight path. This greater surface area may allow for the tract to self-seal relatively easily. The extent of undulation in the tract may be subtle or substantial. Other configurations of tracts (e.g., zig-zag tracts) may also be formed, as suitable for the particular application at hand.

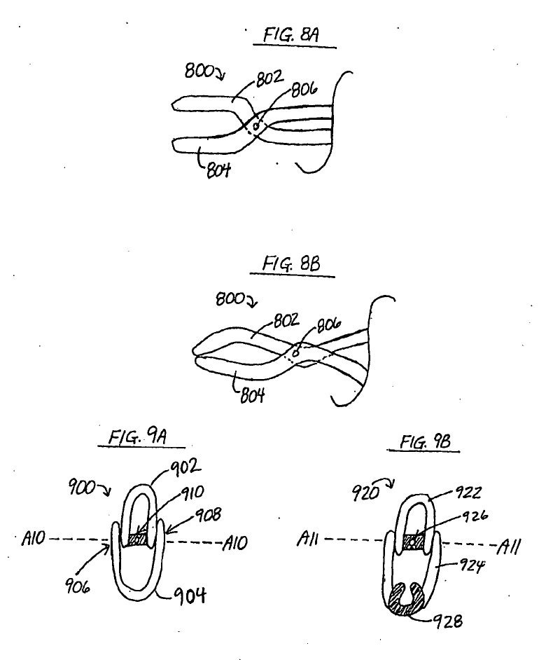

Still further variations of clamping devices may be used to form tracts in tissue. For example, FIGS. 8A and 8B show a device (800) comprising a first clamping arm (802) and a second clamping arm (804) that are connected to each other by a hinge (806). FIG. 8A shows device (800) in a relatively open configuration (e.g., prior to clamping tissue), while FIG. 8B shows device (800) in a relatively closed configuration. Device (800) may be moved into its closed configuration to, for example, clamp tissue between clamping arms (802) and (804). In some variations, device (800) may further comprise one or more tissue-piercing members and/or one or more ports configured to receive a tissue-piercing member, such that the device may be used to form one or more tracts in the tissue that it clamps.

FIGS. 9A and 9B are front views of additional variations of devices for forming tracts in tissue. First, FIG. 9A shows a device (900) comprising two clamping arms (902) and (904) connected to each other via hinges at junction points (906) and (908). Clamping arms (902) and (904) are configured to clamp toward each other around an axis line (A10). Device (900) also includes a port (910) for a tissue-piercing member. FIG. 9B similarly shows a device (920) comprising clamping arms (922) and (924) connected to each other by a hinge and configured to clamp toward each other around an axis line (A11). Device (920) also comprises a port (926) for a tissue-piercing member. As shown in FIG. 9B, a portion of clamping arm (924) has been rolled or otherwise formed into a semi-tubular portion (928) (e.g., having a shape similar to that of a hypotube). The semi-tubular portion may be used, for example, to advance device (920) over a guidewire. While a semi-tubular portion is shown, in some variations, a portion of a clamping arm may be formed into a tubular portion, or into another suitable shape or configuration. Semi-tubular portion (928) may be formed, for example, from rolled sheet metal, or any other appropriate material or materials.

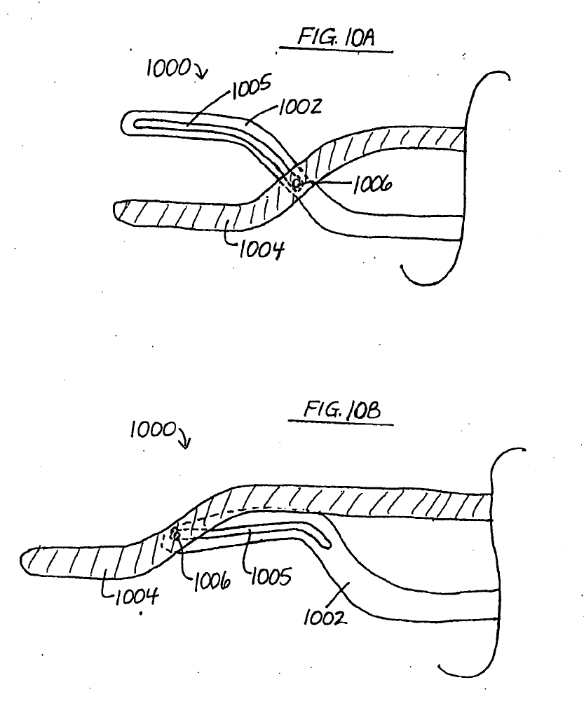

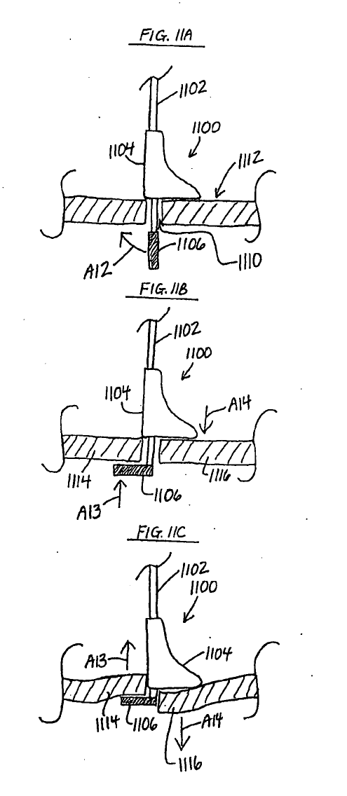

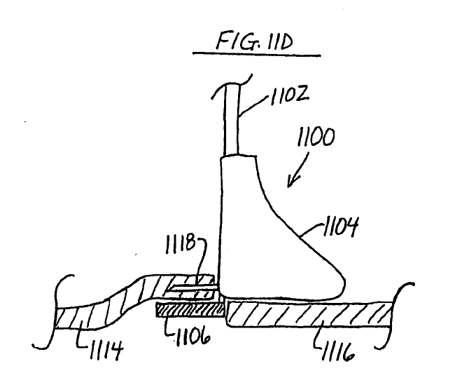

Another variation of a device for forming tracts in tissue is shown in FIGS. 10A and 10B. As shown there, a device (1000) comprises a first clamping arm (1002) that is connected to a second clamping arm (1004). First clamping arm (1002) has a grooved portion (1005), and second clamping arm (1004) has a pin configured to slide within the grooved portion. This allows second clamping arm (1004) to be moved backward, which may, for example, reduce the profile of device (1000) (e.g., during delivery of the device to a target tissue).

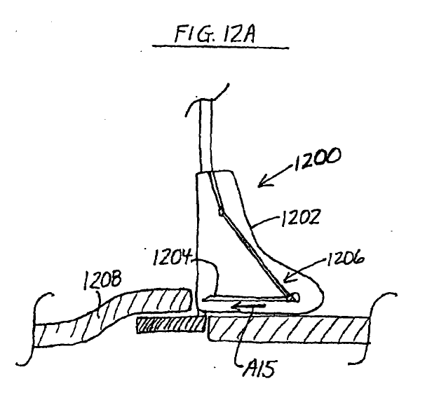

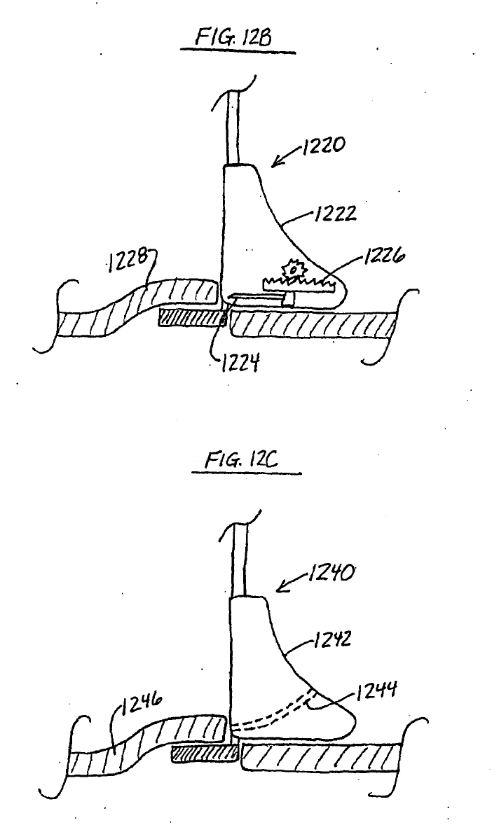

Other types of devices may also be used to clamp, position, and/or manipulate tissue. For example, FIGS. 11A-11D show a method of forming a tract in tissue using a device (1100). As shown in FIG. 11A, device (1100) comprises an elongated member (1102) coupled to a body (1104) and a foot portion (1106). Device (1100) also comprises a tissue-piercing member (1118) configured to be advanced from the body (FIG. 11D). Referring again to FIG. 11A, device (1100) is positioned such that elongated member (1102) extends through an opening (1110) in a tissue wall portion (1112). During use, foot portion (1106) is pivoted in the direction of arrow (A12) and is then pulled up in the direction of arrow (A13) (FIG. 11B), while body (1104) is pushed or held down in the direction of arrow (A14). As a result, and as shown in FIG. 11C, one portion (1114) of tissue wall portion (1112) moves up in the direction of arrow (A13), while another portion (1116) of tissue wall portion (1112) moves down in the direction of arrow (A14). Referring now to FIG. 11D, moving the two portions of the tissue wall portion in different directions helps to position the tissue wall portion for piercing by tissue-piercing member (1118), when it is deployed from body (1104) of device (1100). Other variations of devices (e.g., clamping devices) may also be used to move two or more portions of a tissue wall portion in different directions, as appropriate.

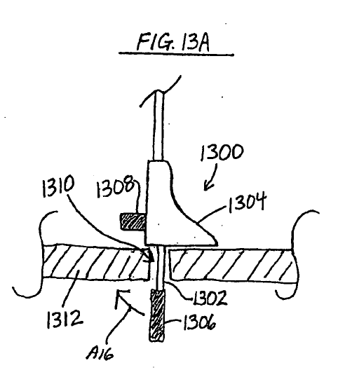

Different mechanisms may be used to deploy a tissue-piercing member from a tissue tract-forming device. As an example, FIG. 12A shows a device (1200) that may be used to form one or more tracts in tissue. As shown there, device (1200) comprises a body (1202) and a tissue-piercing member (1204). Tissue-piercing member (1204) is connected to a linkage mechanism (1206) configured to deploy the tissue-piercing member from the body in the direction of arrow (A15). As the tissue-piercing member is deployed, it may pierce tissue, such as tissue wall portion (1208). As another example, FIG. 12B shows a device (1220) comprising a body (1222) and a tissue-piercing member (1224) configured to form a tract in tissue. Tissue-piercing member (1224) is connected to a gear and rack mechanism (1226) within body (1222). Gear and rack mechanism (1226) is configured to drive tissue-piercing member (1224) from body (1222) into tissue, such as tissue wall portion (1228). While linkage mechanisms and gear and rack mechanisms have been described, other suitable mechanisms or combinations thereof may alternatively or additionally be used to deploy a tissue-piercing member, such as push wires, pull wires, pneumatic mechanisms, hydraulic mechanisms, and the like.

In some variations, a device may be configured to receive a tissue-piercing member that is separate from the device. For example, FIG. 12C shows a device (1240) comprising a body (1242) having a lumen (1244) therethrough. During use, a tissue-piercing member may be advanced through lumen (1244) and into tissue positioned by device (1240), such as the tissue wall portion (1246) shown in FIG. 12C. In certain variations, a device may include both a lumen for receiving a tissue-piercing member and a mechanism for deploying a tissue-piercing member.

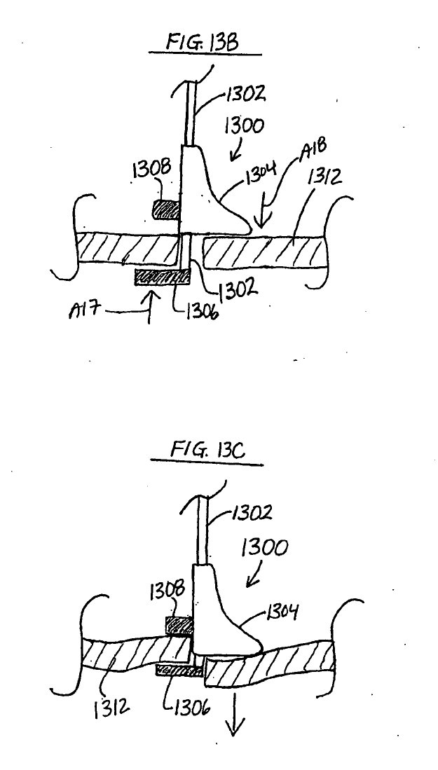

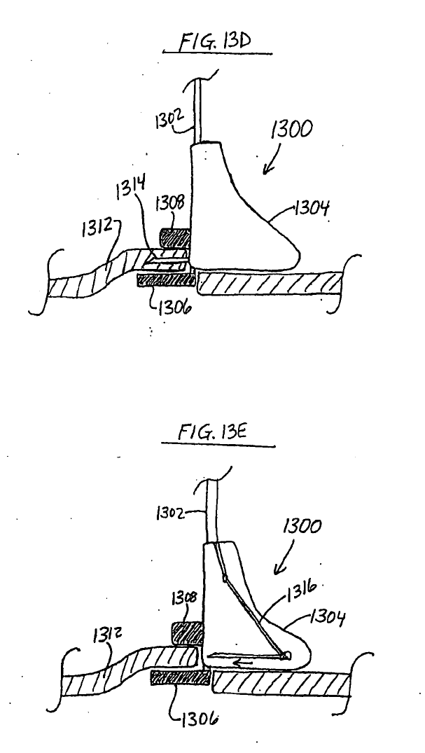

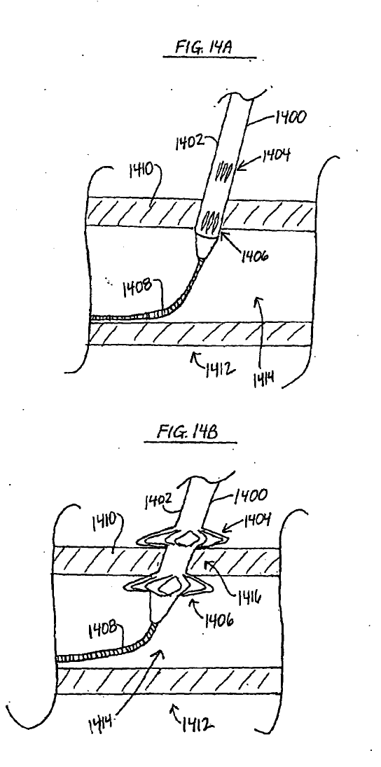

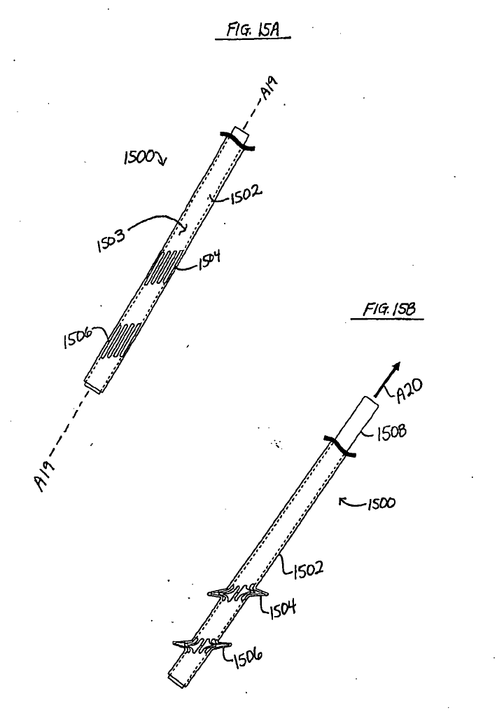

Still other variations of devices may be used for forming one or more tracts in tissue. As an example, FIG. 13A shows a device (1300) comprising an elongated member (1302) coupled to a body (1304) and a foot portion (1306). Device (1300) also comprises a protrusion (1308) on body (1304). As shown in FIG. 13A, device (1300) may be positioned such that elongated member (1302) extends through an opening (1310) in a tissue wall portion (1312). During use, foot portion (1306) may be pivoted in the direction of arrow (A16), and then may be moved upward in the direction of arrow (A17) (FIG. 13B) while body (1304) may be pushed or held down in the direction of arrow (A18). As shown in FIG. 13C, device (1300) may be used to further position tissue wall portion (1312) and then, as shown in FIG. 13D, a tissue-piercing member (1314) may be deployed from body (1304) (e.g., using a linkage mechanism (1316) (FIG. 13E)).

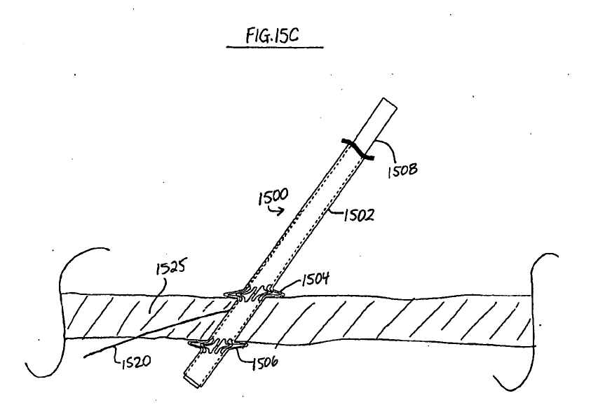

While devices comprising clamping arms have been described, some variations of tissue tract-forming devices may comprise at least one clamping member that is not in the form of a clamping arm. Such variations of devices may also comprise one or more clamping arms, or may not comprise any clamping arms. As an example, FIG. 14A shows a device (1400) comprising a tubular elongated member (1402) having a first expandable region (1404) and a second expandable region (1406). The first and second expandable regions are in the form of multiple slots in elongated member (1402), and are configured, when at least partially expanded, to clamp tissue therebetween. As shown in FIG. 14A, elongated member (1402) may be delivered over a wire (1408) such that the elongated member crosses a tissue wall portion (1410) of an organ (1412) and enters a hollow region (1414) of the organ. FIG. 14B shows device (1400) when first and second expandable regions (1404) and (1406) are expanded so that they clamp a portion (1416) of tissue wall portion (1410) therebetween. This clamping may be used, for example, to position portion (1416) of the tissue wall portion for piercing by one or more tissue-piercing members (not shown) to form one or more tracts in portion (1416). It should be noted that while slots have been described, other suitable openings may alternatively or additionally be used.

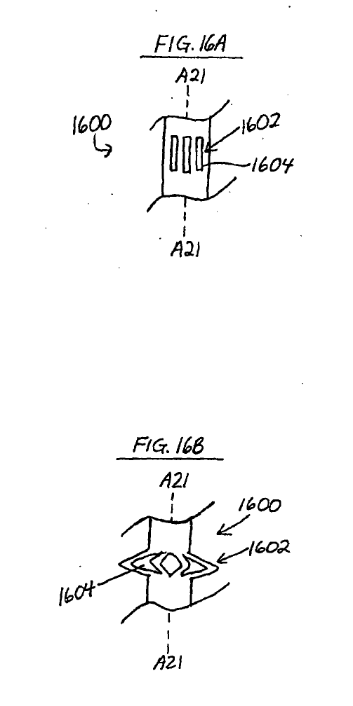

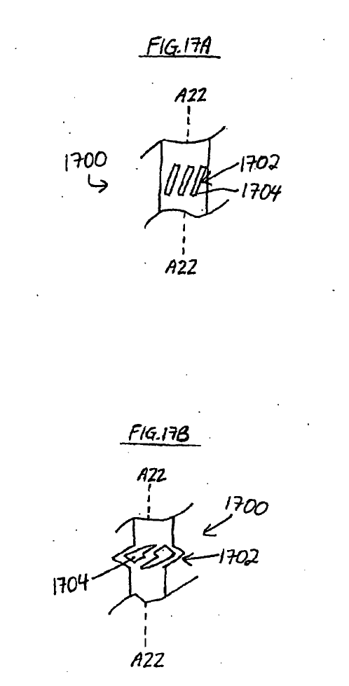

Expandable regions having any suitable size, shape, and configuration may be used. As an example, FIGS. 15A and 15B show a device (1500) comprising an elongated member (1502) having a lumen (1503) and two expandable regions (1504) and (1506). Device (1500) further comprises an inner member (1508) disposed within lumen (1503) of elongated member (1502). Inner member (1508) is configured, when pulled upon, to cause the expandable regions to expand. For example, inner member (1508) may be connected to elongated member (1502) at a point distal to expandable region (1504), such that when inner member (1508) is pulled upon, it causes expandable regions (1504) and (1506) to expand. Inner member (1508) may, for example, be tubular (e.g., such that inner member (1508) may be advanced over a guidewire). In certain variations, inner member (1508) may be used to deliver one or more therapeutic agents to a target site. In some variations, an inner member may have multiple lumens that may be used for different purposes (e.g., one for therapeutic agent delivery and another for advancement over a guidewire). Other suitable push and/or pull mechanisms may alternatively or additionally be used to expand one or more expandable regions of a device.

Expandable regions (1504) and (1506) are configured such that when they are expanded (FIG. 15B), they are angled relative to the longitudinal axis (A19) (FIG. 15A) of elongated member (1502). This angling of the expanded expandable regions relative to the longitudinal axis may, for example, cause the elongated member to assume a desired angle relative to a tissue portion being clamped between the expandable regions. This, in turn, may provide for a desired angled entry of a tissue-piercing member from the elongated member into the tissue portion. For example, FIG. 15C shows a tissue-piercing member (1520) being deployed from elongated member (1502) (e.g., through a channel, port, or opening in the elongated member) and into a tissue wall (1525), when elongated member (1502) has been positioned in the tissue wall using expandable regions (1504) and (1506). In some variations, expandable regions (1504) and (1506) may cause local tissue distortion that helps to orient tissue-piercing member (1520) in a desired orientation when it is deployed into the tissue.

As shown in FIG. 15B, expandable regions (1504) and (1506) may be expanded by pulling on inner member (1508) in the direction of arrow (A20). However, other mechanisms for expanding the expandable regions may alternatively or additionally be used. Moreover, while the above figures illustrate the expansion of both expandable regions of a device, in certain variations, a method may comprise expanding one or more expandable regions of a device without expanding one or more other expandable regions of the device.

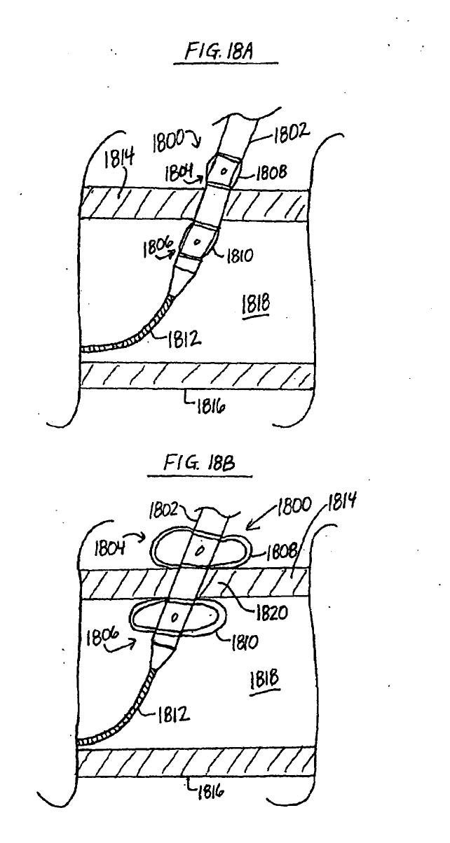

Any suitable configurations of slits and/or other openings may be used in an expandable region. As an example, FIGS. 16A and 16B show a portion of an elongated member (1600) comprising an expandable region (1602) comprising slits (1604) that are parallel to the longitudinal axis (A21) of the elongated member. FIG. 16A shows expandable region (1602) prior to expansion, while FIG. 16B shows expandable region (1602) after it has been expanded. As another example, FIGS. 17A and 17B show a portion of an elongated member (1700) comprising an expandable region (1702) comprising slits (1704) that are angled relative to the longitudinal axis (A22) of the elongated member. FIG. 17A shows expandable region (1702) prior to expansion, while FIG. 17B shows expandable region (1702) after it has been expanded. Any number of slits or other openings may be used in an expandable region. For example, some variations of expandable regions may include relatively few (e.g., two, three) slits and/or other openings, while other variations of expandable regions may include more (e.g., five, ten) slits and/or other openings. Moreover, in certain variations, a device may include one expandable region having a certain number of slits and/or other openings, and another expandable region having a different number of slits and/or other openings. The slits and/or other openings in an expandable region may be of any suitable size or shape, and different combinations of different types of slits and/or other openings may sometimes be used. In some variations, an expandable region may be at least partially coated (e.g., with silicone). This may, for example, cover any slits and/or other openings in the expandable region (e.g., to create a solid seal).

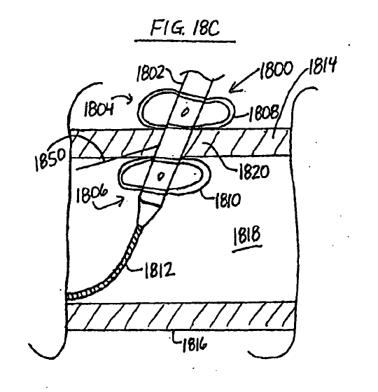

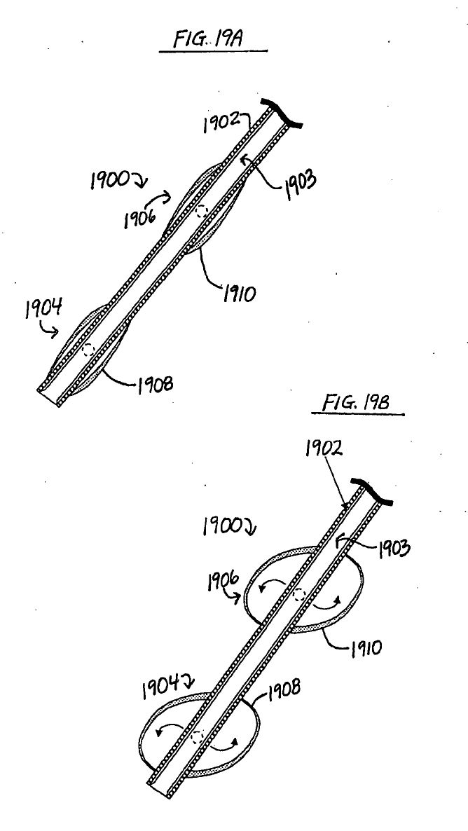

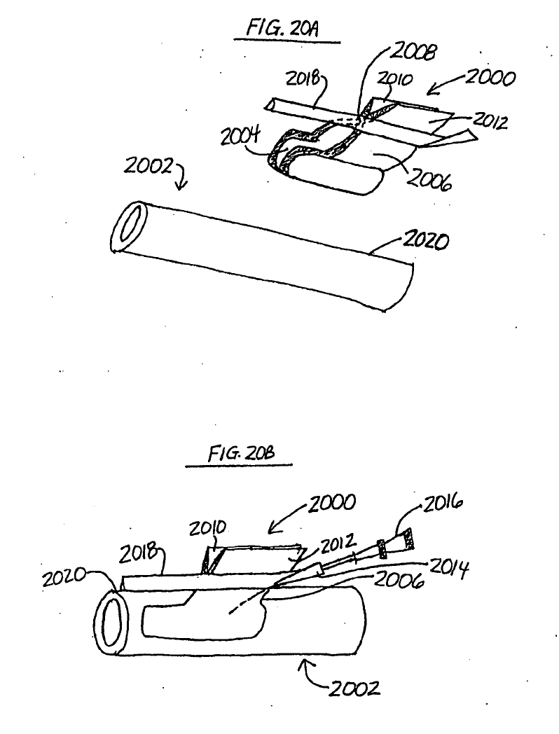

In certain variations, an expandable region may comprise an inflatable member. For example, FIGS. 18A-18C show a device (1800) comprising an elongated member (1802) having two expandable regions (1804) and (1806) comprising inflatable members (1808) and (1810). FIG. 18A shows device (1800) being delivered over a wire (1812), across a tissue wall portion (1814) of an artery (1816) and into a lumen (1818) of the artery. FIG. 18B shows device (1800) after inflatable members (1808) and (1810) have been inflated (e.g., by flowing inflation fluid through a lumen of the elongated member) to clamp a portion (1820) of tissue wall portion (1814) between them. FIG. 18C shows a tissue-piercing member (1850) being deployed from elongated member (1802) (e.g., through a channel, port, or opening in the elongated member), through tissue wall portion (1814), and into lumen (1818). The tissue-piercing member thereby forms a tract within the tissue wall portion.

While FIGS. 18A-18C show a device comprising two expandable regions, any suitable number of expandable regions may be employed. For example, a device may have more than two expandable regions, such as three, four, five, or six expandable regions. The expandable regions may be roughly the same size when expanded, or may be different sizes when expanded. Moreover, when multiple expandable regions are used, the expandable regions may be the same type of expandable region, or may be different types of expandable regions.

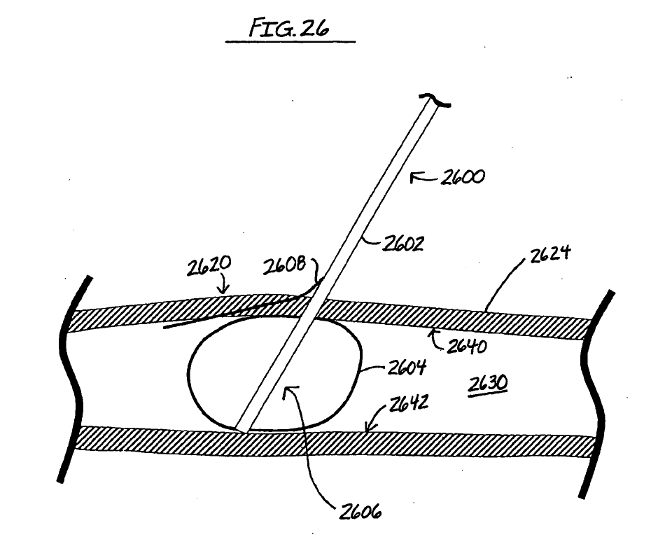

In some variations, a device may comprise just one expandable region, such as an inflatable member, that may be used, for example, to help position and/or isolate tissue. As an example, FIG. 26 shows a device (2600) comprising an elongated member (2602) and an inflatable member (2604) coupled to a distal portion (2606) of the elongated member. Device (2600) also comprises a tissue-piercing member (2608) configured to be deployed from elongated member (2602). FIG. 26 shows device (2600) after inflatable member (2604) has been inflated (e.g., by flowing inflation fluid through a lumen of the elongated member), to help position a vessel wall portion (2620) of a vessel (2624) for piercing by tissue-piercing member (2608). In some variations, inflatable member (2604) may be slightly over-inflated, thereby providing a highly stabilized isolated portion of tissue for tract formation. FIG. 26 depicts tissue-piercing member (2608) after it has been deployed from elongated member (2602), through vessel wall portion (2620), and into a lumen (2630) of vessel (2624). The tissue-piercing member thereby forms a tract within vessel wall portion (2620).

As noted above, inflatable member (2604) may help to position vessel wall portion (2620) for piercing by tissue-piercing member (2608). For example, the inflatable member may position the vessel wall portion so that the tissue-piercing member enters the vessel wall portion at a specific angle. In addition to helping position vessel wall portion (2620), inflatable member (2604) may help to stabilize device (2600) during use (e.g., by temporarily anchoring the device at the target site). For example, and as shown, the inflatable member may contact opposing lumen wall surfaces (2640) and (2642) of vessel (2624). This may help to prevent device (2600) from slipping or otherwise becoming displaced or moved out of position. While a specific inflatable member has been shown, any suitable expandable region may be employed including, without limitation, donut-shaped inflatable members, hoops or rings (including, e.g., multi-wire hoops), and stents or stent-like structures.

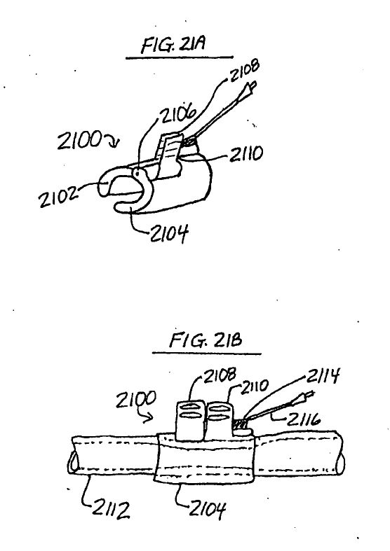

Inflatable members may, when inflated, be symmetrically disposed relative to an elongated member, or eccentrically disposed relative to an elongated member. For example, FIG. 19A shows a device (1900) comprising an elongated member (1902) and two expandable regions (1904) and (1906) comprising inflatable members (1908) and (1910), respectively. Elongated member (1902) has a double-walled lumen (1903) that is in fluid communication with inflatable members (1908) and (1910). As shown in FIG. 19B, once inflated (e.g., by flowing an inflation fluid such as saline through lumen (1903)), inflatable members (1908) and (1910) are eccentrically disposed on elongated member (1902). Inflatable members (1908) and (1910) each have walls of asymmetric thickness that cause the inflatable members to be eccentric when inflated.

Eccentric inflatable members may be configured in any of a number of different ways. In some variations, an inflatable member may be configured to deploy in a tilted manner. For example, the inflatable member may have a wall of varying thickness to provide uneven oblong inflation, or the inflatable member may be mounted such that it is tilted on the elongated member. Furthermore, in certain variations, an inflatable member may be rendered eccentric by using a pullwire to distort the shape of the inflatable member. Such a pullwire may be used, for example, if manufacturing an eccentric inflatable member would be relatively expensive, and/or if an eccentric inflatable member would be relatively difficult to reliably and/or reproducibly manufacture.

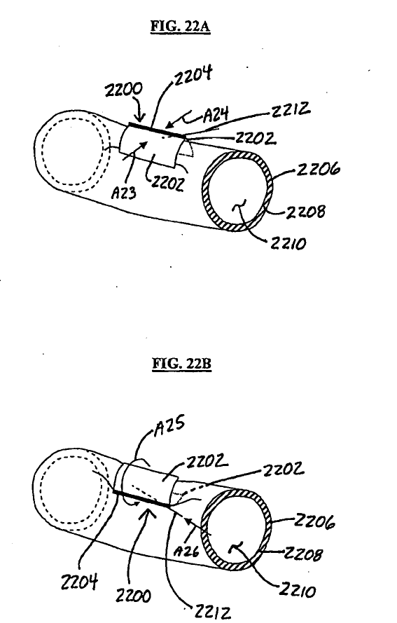

In some variations, a device for forming one or more tracts in tissue may comprise curved surfaces that are configured to clamp tissue therebetween. As an example, FIG. 20A shows a device (2000) and a portion of a vessel (2002). As shown there, device (2000) comprises a first curved surface (2004) and a second curved surface (2006) opposed to the first curved surface. The curved surfaces are coupled to each other at an attachment point (2008). Device (2000) also includes wings (2010) and (2012) for actuating the first and second curved surfaces to move them about attachment point (2000) between a collapsed position and an outwardly displaced position. In some variations, device (2000) may have a spring-loaded clamping mechanism.

In FIG. 20A, the first and second curved surfaces are in their outwardly displaced position, as device (2000) is approaching vessel (2002). In certain variations, device (2000) may be delivered to vessel (2002) directly via an incision in the skin that is cut down to the vessel. As shown in FIG. 20B, upon arrival at vessel (2002), the first and second curved surfaces are in their collapsed position, so that they clamp vessel (2002). Device (2000) further comprises a tissue-piercing member port or lumen (2014). During use, and as shown in FIG. 20B, a tissue-piercing member (2016) may be advanced through the port or lumen and into a wall of vessel (2002) (e.g., following the pathway shown in a broken line in FIG. 20B). The tissue-piercing member may be advanced into the vessel wall at any suitable angle relative to the longitudinal axis of the vessel. In some variations, the port or lumen may be configured to achieve a specific angle of advancement. In certain variations, the desired angle of advancement of the tissue-piercing member may become steeper as the tissue wall becomes thicker. Device (2000) additionally comprises an alignment surface (2018) that helps to align the device along an exterior surface (2020) of vessel (2002). However, devices without alignment surfaces may also be used.

A device such as device (2000) may be sized and configured to clamp entirely around a vessel, or to clamp only a selected portion of a vessel. In some variations in which a device clamps entirely around a vessel, the device may cause the vessel to temporarily collapse. A tissue-piercing member may then be used to form a tract through the vessel wall (e.g., in a period of about 5 seconds or less). A device such as device (2000) may, for example, be used to stabilize a tissue portion for ease of deployment of a tissue-piercing member through the tissue portion.

FIGS. 21A and 21B show another variation of a device for forming one or more tracts in tissue. As shown there, a device (2100) comprises curved members (2102) and (2104) connected to each other by a hinge (2106). Device (2100) also comprises wings (2108) and (2110) for moving curved members (2102) and (2104) between a collapsed position and an outwardly displaced position. As shown in FIG. 21B, device (2100) may be used to substantially or entirely encircle a vessel (2112) (or another portion of tissue). This may help to capture vessel (2112) as an intact vessel. Device (2100) includes a port or lumen (2114) through which a tissue piercing member (2116) may be advanced so that the tissue-piercing member can form a tract through a wall portion of vessel (2112). The angle of advancement of the tissue-piercing member through the port of lumen may be fixed or adjustable.

In some variations, one or more of the devices and/or methods described here may be used to form one or more tracts in rotated tissue. For example, a method may comprise using a device to clamp at least a portion of a tissue wall, rotating the portion of the tissue wall (e.g., using the device), and advancing a tissue-piercing member through the rotated tissue to form the tract. The rotating may help to position the tissue-piercing member relative to the tissue wall. Tissue rotation may be particularly desirable, for example, when an initial Seldinger stick is performed off the center-axis. The tissue may be rotated in either direction about a tissue circumference (e.g., from 0°-360°, from 0°-180°, from 0°-45°, from 45°-90°, etc.). However, the tissue need not be rotated a significant amount (e.g., the tissue may be rotated 1°, 5°, 10°, 15°, etc.) and the entire tissue thickness need not be rotated.

FIGS. 22A and 22B provide an illustrative depiction of a clamping device (2200) being used to rotate tissue and to form a tract in the rotated tissue. As shown there, clamping device (2200) comprises wings (2202) connected by a hinge portion (2204). In use, wings (2202) may be clamped together (arrows (A23) and (A24)) so that they clamp around a portion of a vessel (2206) having a wall portion (2208) and a lumen (2210). Referring specifically to FIG. 22A, a tissue-piercing member (2212) is configured to be advanced through the clamping device (e.g., through a port or lumen in the clamping device). The tissue-piercing member may further be advanced into tissue being clamped by the clamping device.

As shown in FIG. 22B, clamping device (2200) may be rotated in the direction of arrow (A25), thereby rotating the clamped portion of vessel (2206). Tissue-piercing member (2212) may then be advanced through the clamping device in the direction of arrow (A26) and into wall portion (2208) of vessel (2206), to form a tract in the wall portion. Alternatively or additionally, the tissue-piercing member may be advanced prior to and/or during rotation of the clamped portion of vessel (2206). In some variations, a clamped portion of tissue may only be rotated once, while in other variations, it may be rotated multiple times (e.g., in the same direction or in different directions). The clamped portion of tissue may also be otherwise manipulated (e.g., tented). Moreover, while tissue-piercing member (2212) is depicted as part of clamping device (2200), some variations of methods may comprise using one or more tissue-piercing members that are separate from a device that is used to isolate, immobilize, and/or position tissue for tract formation. Additionally, it should be noted that other variations of devices described here may also be used to rotate tissue, as appropriate.

Rotation of tissue prior to and/or during tract formation may be useful to effect a desirable tissue-piercing member location, which may in turn be useful for forming a tract having suitable thicknesses of tissue on either side. This may help ensure that the tract is robust enough to withstand repetitive insertion of various tools. In addition, having sufficient tissue thickness on either side of the tract may help the tract seal more quickly. Initial positioning of the tissue-piercing member away from one or more surfaces of the tissue wall may also help with the formation of a longer tract, which may be useful in ensuring more rapid sealing.

Of course, rotation of tissue may be used as an alternative to, or in addition to, one or more other methods of tissue manipulation, such as tissue tenting, tissue deformation, and the like. In some variations, devices and/or methods described herein may be used in conjunction with device and/or methods of applying a vacuum to tissue. Certain variations of the devices described here may comprise at least one suction member configured for connection to one or more vacuum sources. For example, a device may comprise a clamping arm comprising a suction member. The clamping arm may be configured to clamp tissue, and also to suction the tissue (e.g., to enhance the tissue-clamping). In variations in which the device comprises at least one suction member, the device may have one or more lumens, slots, holes, openings, etc. for facilitating connection of the suction member to a vacuum source. Methods of manipulating tissue and/or applying a vacuum to tissue are described, for example, in U.S. patent application Ser. Nos. 11/873,957 (published as US 2009/0105744 A1) and 61/082,449, both of which were previously incorporated herein by reference in their entirety.

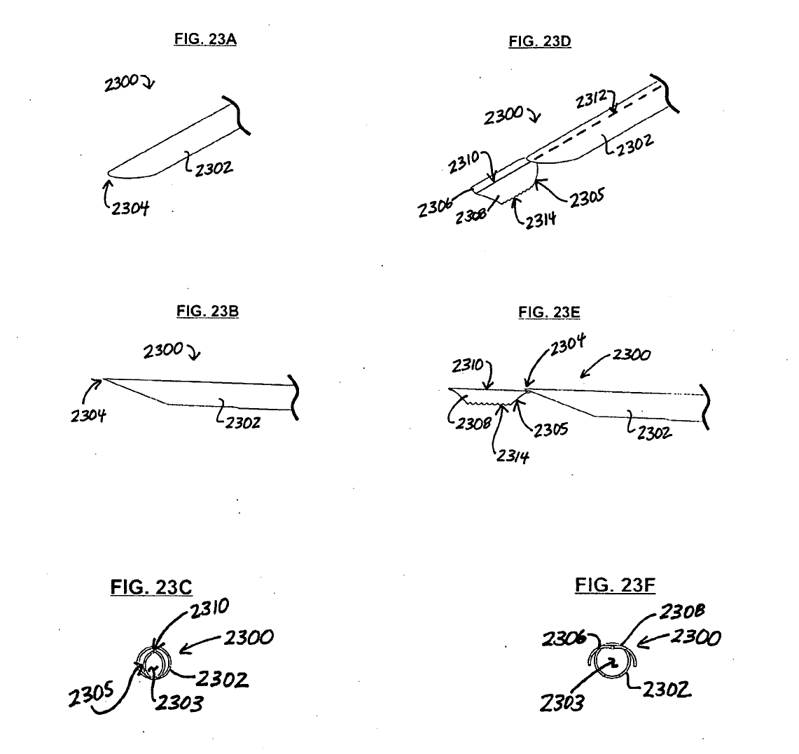

FIGS. 23A-23F depict another variation of a clamping device (2300). As shown there, clamping device (2300) comprises an outer tissue-piercing member (2302), such as a trocar, having a beveled tissue-piercing tip (2304) and a lumen (2303) (FIGS. 23C and 23F). Clamping device (2300) also comprises a clamping member (2305) slidably disposed within lumen (2303). Clamping member (2305) comprises two clamping portions (2306) and (2308) coupled to each other at a hinge region (2310). Clamping portions (2306) and (2308) are also coupled to a push-pull member (2312) (shown only in FIG. 23D), which may be used to slidably move clamping member (2305) within, and at least partially outside of, lumen (2303). Push-pull member (2312) may, for example, be in the form of a push-pull wire.

Clamping device (2300) may also comprise at least one additional tissue-piercing member (not shown) that may be used to form one or more tracts in a target tissue. In some variations, the additional tissue-piercing member may be slidably disposed within lumen (2303) of outer tissue-piercing member (2302). Alternatively or additionally, clamping device (2300) may be configured to receive and/or position at least one additional tissue-piercing member that is separate from clamping device (2300).

In FIGS. 23A-23C, clamping portions (2306) and (2308) are disposed within lumen (2303) of outer tissue-piercing member (2302). This configuration of clamping device (2300) may be used, for example, during advancement of the clamping device to a target site, such as a target vessel, and may limit the likelihood of the clamping portions becoming caught on, and/or damaging, tissue on the way to the target site. Clamping portions (2306) and (2308) may be withdrawn into lumen (2303) by, for example, pulling on a proximal portion of push-pull member (2312). Alternatively or additionally, outer tissue-piercing member (2302) may be distally advanced over clamping portions (2306) and (2308). When clamping portions (2306) and (2308) are disposed within lumen (2303), they may be relatively constrained, as shown in FIG. 23C. Clamping device (2300) may be advanced through tissue surrounding a target site with the help of, for example, tissue-piercing tip (2304) of outer tissue-piercing member (2302), which may cut a path through the surrounding tissue. In some variations, the surrounding tissue may provide an additional force that pushes down on clamping portions (2306) and (2308) while clamping device (2300) is being advanced to the target site.

In FIGS. 23D-23F, clamping portions (2306) and (2308) have been deployed from lumen (2303) of outer tissue-piercing member (2302) (e.g., by pushing on a proximal portion of push-pull member (2312)). Such deployment may occur, for example, upon reaching a target site (e.g., when tissue clamping is desired). As shown in FIG. 23F, clamping portions (2306) and (2308) may become less constrained as they exit lumen (2303) of outer tissue-piercing member (2302), effectively springing apart from each other around hinge region (2310). Clamping portions (2306) and (2308) may then be positioned around a desired clamping region, and may be actuated to clamp the region. In some variations, an actuator of clamping device (2300), such as a button or slide actuator, may be used to actuate the clamping portions. Alternatively or additionally, certain components of clamping device (2300) may be moved relative to each other to actuate the clamping member, as described in additional detail below.

As shown in FIGS. 23D and 23E, clamping portion (2308) has a serrated gripping edge (2314). Clamping portion (2306) may also have a serrated gripping edge, or may have a different configuration. Moreover, other variations of clamping portions may have still other configurations, as appropriate. Referring back to FIGS. 23D and 23E, serrated gripping edge (2314) may, for example, help to enhance the clamping and retention of tissue at a target site.

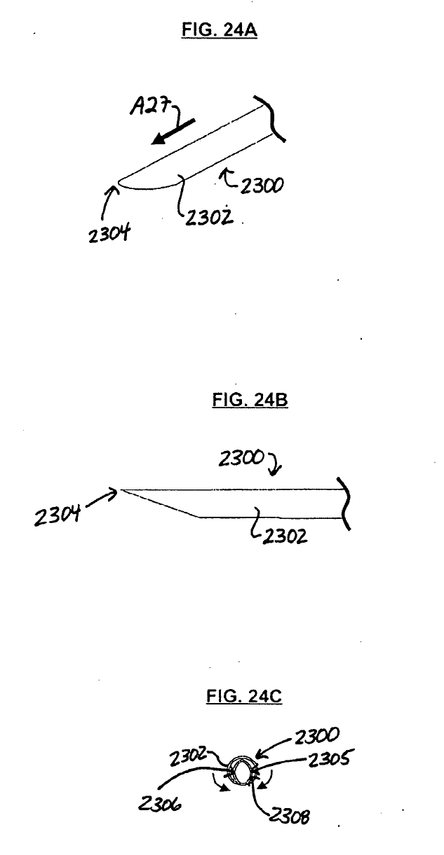

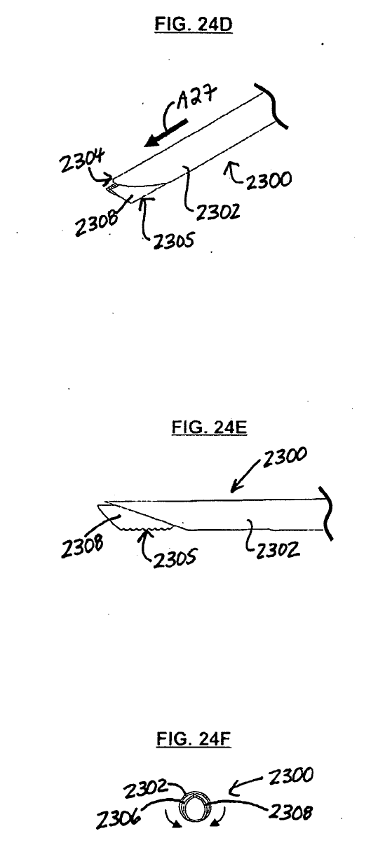

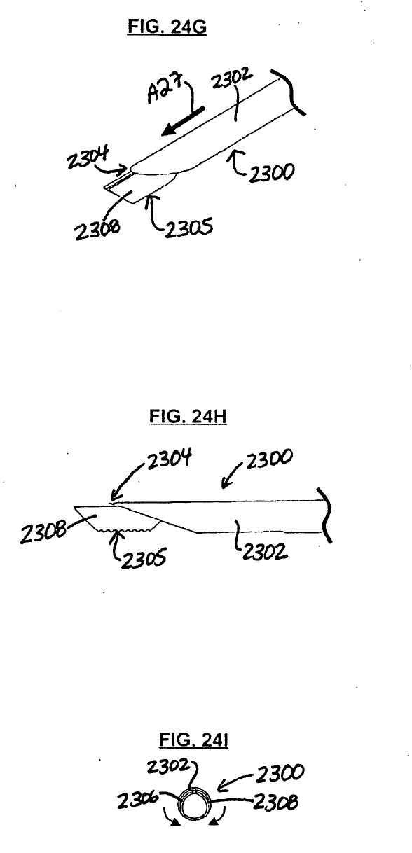

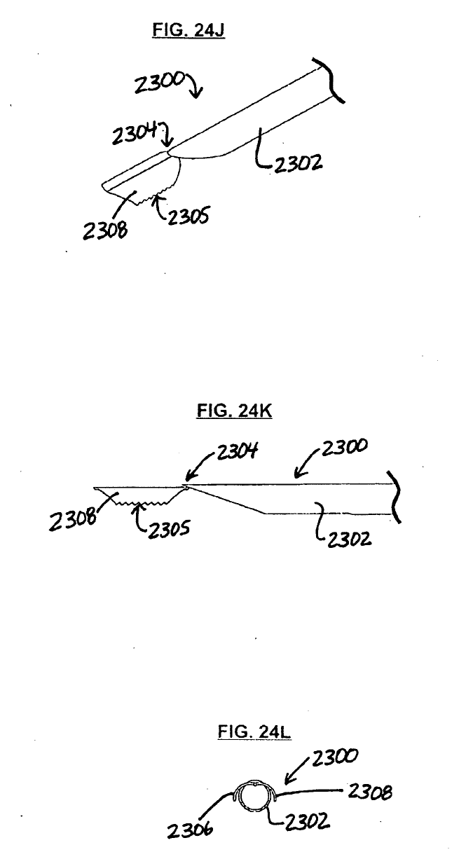

FIGS. 24A-24L illustrate the various positions that may be assumed by clamping device (2300) during deployment of clamping member (2305).

First, in FIGS. 24A-24C, clamping member (2305) is completely withdrawn within lumen (2303) of outer tissue-piercing member (2302). However, an operator may begin to advance push-pull member (2312) distally, in the direction of arrow (A27), to initiate the deployment process. As shown in FIG. 24C, when clamping member (2305) is completely disposed within lumen (2303), the walls of outer tissue-piercing member (2302) exert a constraining force on clamping portions (2306) and (2308). As described above, this position may be especially well-suited for advancement of clamping device (2300) to a target site. In some variations, clamping device (2300) may be advanced to a target site using one or more imaging techniques, such as ultrasound, and/or using one or more localization techniques (e.g., by measuring blood flow with vascular Doppler).

FIGS. 24D-24F show clamping device (2300) after the operator has started deploying clamping member (2305) from lumen (2303) of outer tissue-piercing member (2302). While clamping portions (2306) and (2308) are now located partially outside of lumen (2303), they are also partially within the lumen. As a result, the walls of outer tissue-piercing member (2302) still exert a constraining force on clamping portions (2306) and (2308), as shown in FIG. 24F.

In FIGS. 24G-24I, clamping member (2305) has been pushed even farther in the direction of arrow (A27), although the walls of outer tissue-piercing member (2302) still exert a constraining force on clamping portions (2306) and (2308), as shown in FIG. 24I.

Finally, and referring to FIGS. 24J-24L, once clamping member (2305) has been pushed out of lumen (2303), the walls of outer tissue-piercing member (2302) no longer exert a constraining force on clamping portions (2306) and (2308). As a result, the clamping portions open away from each other (FIG. 24L), and are ready to be positioned for clamping tissue. In some cases, clamping portions (2306) and (2308) may comprise one or more springs therebetween that bias the clamping portions apart from each other in the absence of a sufficiently large constraining force. In certain variations, a spring force may effect the opening of clamping portions (2306) and (2308), and may result in the formation of a dissection plane in tissue surrounding the target tissue. Clamping portions (2306) and (2308) may then be positioned on the target tissue surface and may be used to clamp at least a portion of the target tissue.

As described above, in some variations, clamping may be effected by an actuation mechanism that closes clamping portions (2306) and (2308) toward each other. Alternatively or additionally, clamping may be effected by proximally withdrawing clamping member (2305) at least partially into lumen (2303) of outer tissue-piercing member (2302), and/or distally advancing outer tissue-piercing member (2302) over clamping member (2305), and thereby causing clamping portions (2306) and (2308) to close toward each other. In some variations, the degree of clamping may be controlled by controlling the withdrawal of clamping member (2305) into lumen (2303) and/or the advancement of outer tissue-piercing member (2302) over clamping member (2305).

Once the tissue has been clamped, one or more tissue-piercing members may be advanced into the clamped tissue. For example, a tissue-piercing member may be advanced between clamping portions (2306) and (2308) and into the clamped tissue, to form a tract in the tissue. Other suitable tissue-piercing member advancement pathways may alternatively or additionally be used, as appropriate.

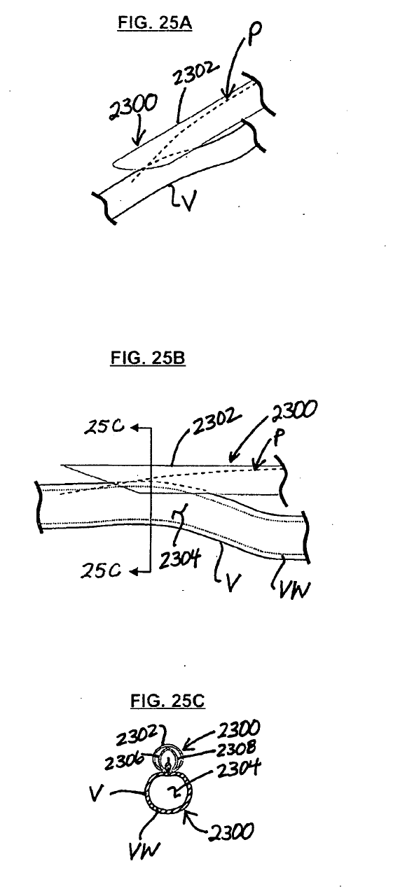

FIGS. 25A-25C provide an illustrative depiction of clamping device (2300) being used to clamp a portion of a vessel wall (VW) of a vessel (V), such as an artery. FIGS. 25A and 25B also show a projected path for a tissue-piercing member that may, for example, be deployed from outer tissue-piercing member (2302) and into vessel wall (VW), to form a tract in the vessel wall. Once the desired tract has been formed (and, in some cases, once one or more tools have been advanced through the tract and/or one or more procedures have been performed), clamping portions (2306) and (2308) may be actuated such that they open up and release the clamped portion of tissue. Clamping device (2300) may then be removed from the body.

In certain variations of tissue tract-forming methods, at least one component of a device that is used to clamp or otherwise isolate or position a portion on tissue may not contact the portion of tissue when the device is in use. As an example, a method may comprise using first and second clamping arms of a clamping device to clamp a portion of tissue, where the first clamping arm contacts the portion of tissue, while the second clamping arm does not contact the portion of tissue. For example, the second clamping arm may contact either a skin surface, or tissue that is located between the portion of tissue and a skin surface. As an example, the first clamping arm may be a distal or lower clamping arm that is delivered into a vessel lumen, and that contacts the lumen wall. The second clamping arm may be an upper or proximal clamping arm that is not delivered into the body. Rather, the second clamping arm may contact a skin surface of the body. The two clamping arms may then be clamped toward each other, such that the portion of tissue is clamped therebetween (even though one of the clamping arms does not contact the portion of tissue). Keeping the second clamping arm external to the skin surface may, for example, allow for a relatively low-profile first clamping arm to be delivered into the body (e.g., such that the operator can initiate and complete a procedure relatively easily and efficiently). It should be understood that any suitable devices described herein may be used to clamp or otherwise isolate or position a portion of tissue in this manner.

In some variations, a component of a device may, for example, include one or more relatively soft features for contacting a skin surface. As an example, a component of a device may include an inflatable member, such as a relatively soft balloon, that contacts a skin surface when the device is in use. Alternatively or additionally, a component of a device may comprise one or more springs that contact a skin surface when the device is in use (e.g., to provide sufficient tension against the skin surface for isolating a portion of tissue).

Some variations of the devices described here may comprise one or more heating elements, electrodes, and/or sensors (e.g., Doppler, pressure, nerve sensors, ultrasound sensors, etc.), one or more drug delivery ports along a surface thereof, one or more radiopaque markers to facilitate visualization, or the like. In certain variations in which a device comprises one or more sensors, the device may be used to sense at least one useful parameter, such as temperature, pressure, tissue identification or location (e.g., nerves or various anatomical structures), and/or blood flow within a vessel. For example, if the parameter is blood flow within a vessel, the device may be repositioned if blood flow within a vessel is detected.

In some variations, the devices may comprise one or more energy applicators, and may be used to apply energy to tissue. This may, for example, help to seal the tissue. The energy may come from any suitable energy source (e.g., energy selected from the group consisting of ultrasound, radiofrequency (RF), light, magnetic, or combinations thereof).

Certain variations of the devices may comprise one or more cameras (e.g., to facilitate direct visualization). The camera may or may not have a corresponding light or illumination source, and may be included at any suitable location on the device.

In some variations, kits may incorporate one or more of the devices and/or device components described here. In certain variations, the kits may include one or more of the devices for forming a tract through tissue described here, one or more of the device components described here (e.g., tissue-piercing members), and/or one or more additional tools. For example, the tools may be those that are advanced through the tract during the performance of a procedure (e.g., guide wires, scissors, grippers, ligation instruments, etc.), one or more supplemental tools for aiding in closure (e.g., an energy delivering device, a closure device, and the like), one or more tools for aiding in the procedure (e.g., gastroscope, endoscope, cameras, light sources, etc.), combinations thereof, and the like. Of course, instructions for use may also be provided with the kits.

In some variations, one or more tracts may be formed in a tissue having one or more irregular tissue surfaces. The irregular surfaces may be in the form of, for example, undulations, bends, curves, recesses, protrusions, any combination of these, or the like. Methods of forming tracts in irregular tissue surfaces are described, for example, in U.S. patent application Ser. No. 11/873,957 (published as US 2009/0105744 A1), which was previously incorporated by reference in its entirety.

While the above devices and methods have been described for use in forming one or more tracts in tissue, in some variations, one or more of the above-described devices and/or methods may be used for one or more other purposes. As an example, a device and/or method may be used to position a selected portion of tissue for delivery of a therapeutic agent into that portion of tissue, without also forming a tract in the tissue.

While the devices and methods have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims.

What is claimed is:

1. A device for forming a tract in tissue comprising:
a clamping member comprising a first elongated clamping arm and a second elongated clamping arm configured to clamp tissue therebetween; and
a tissue-piercing member configured to form a tract in at least a portion of the clamped tissue.

2. The device of claim 1, further comprising a housing.

3. The device of claim 2, wherein the tissue-piercing member is slidably disposed within the housing.

4. The device of claim 1, wherein the first and second elongated clamping arms are coupled to each other by a hinge.

5. The device of claim 1, wherein the tissue-piercing member is a needle.

6. The device of claim 5, wherein the needle is hollow.

7. A method for forming a tract in tissue of a subject comprising:
clamping at least a portion of tissue between a first elongated clamping arm and a second elongated clamping arm of a clamping member; and
advancing a tissue-piercing member in a first direction through at least a portion of the clamped tissue to form a tract in the tissue.

8. The method of claim 7, wherein the first elongated clamping arm contacts the portion of tissue while the second elongated clamping arm does not contact the portion of tissue.

9. The method of claim 8, wherein the second elongated clamping arm contacts a skin surface of the subject.

10. The method of claim 7, further comprising advancing the clamping member through an opening in the tissue prior to clamping at least a portion of the tissue with the clamping member.

11. The method of claim 10, wherein advancing the clamping member through the opening in the tissue comprises advancing the clamping member over a guidewire.

12. The method of claim 7, wherein the tissue is tissue of a vessel wall.

13. The method of claim 12, wherein the tissue is tissue of an arterial wall.

14. The method of claim 7, wherein the tissue comprises an organ.

15. The method of claim 14, wherein the organ is selected from the group consisting of an organ of the cardiovascular system, an organ of the digestive system, an organ of the respiratory system, an organ of the excretory system, an organ of the reproductive system, and an organ of the nervous system.

16. The method of claim 14, wherein the organ is an organ of the digestive system.

17. The method of claim 16, wherein the organ is a stomach.

18. The method of claim 7, wherein the tissue-piercing member enters the clamped tissue at a first location, and exits the clamped tissue at a second location, and wherein the length between the first and second locations is greater than the thickness of the tissue.

19. The method of claim 7, wherein the length of the tract is greater than the thickness of the tissue.

20. The method of claim 7, further comprising advancing one or more tools through the tract.

21. The method of claim 7, further comprising withdrawing the tissue-piercing member from the tissue.

22. The method of claim 21, wherein the tract self-seals after the tissue-piercing member has been withdrawn from the tissue.

23. The method of claim 22, wherein the tract self-seals within 15 minutes or less.

24. The method of claim 22, wherein the tract self-seals within 5 minutes or less.

25. The method of claim 22, wherein the tract self-seals within 1 minute or less.

26. A device for forming a tract in tissue comprising:
an elongated member;
a body coupled to the elongated member;
a foot portion coupled to the elongated member; and
a tissue-piercing member configured to be advanced from the body, wherein the body is configured to displace a first portion of a tissue in a first direction and the foot portion is configured to displace a second portion of the tissue in a second direction that is different from the first direction.

27. The device of claim 26, wherein the second direction is opposite the first direction.

28. The device of claim 26, wherein the tissue-piercing member is a needle.

29. A method for forming a tract in tissue comprising:
contacting a tissue with a device comprising an elongated member, a body coupled to the elongated member, and a foot portion coupled to the elongated member to displace a first portion of the tissue in a first direction and a second portion of the tissue in a second direction that is different from the first direction; and
advancing a tissue-piercing member through the displaced second portion of the tissue to form a tract in the tissue.

30. A device for forming a tract in tissue comprising:
a clamping member comprising a first expandable region and a second expandable region configured to clamp tissue therebetween; and
a tissue-piercing member configured to form a tract in at least a portion of the clamped tissue.

31. The device of claim 30, wherein the first expandable region comprises a first inflatable member.

32. The device of claim 31, wherein the second expandable region comprises a second inflatable member.

33. The device of claim 30, wherein the tissue-piercing member is a needle.

34. A method for forming a tract in tissue comprising:
clamping tissue between a first region of a clamping member and a second region of the clamping member when each of the first and second regions is in an expanded configuration; and
advancing a tissue-piercing member through at least a portion of the clamped tissue to form a tract in the tissue.

35. A device for use in forming tissue tracts comprising:
a first curved surface; and
a second curved surface opposed to the first curved surface and coupled to the first curved surface at an attachment point, the first and second curved surfaces configured to move about the attachment point between a first collapsed position and a second outwardly displaced position,
wherein the device defines a lumen configured to receive a tissue-piercing member, and wherein the first and second curved surfaces, when in the first collapsed position, are configured to clamp tissue and to position at least a portion of the tissue for piercing by a tissue-piercing member passing through the lumen.

36. The device of claim 35, further comprising a tissue-piercing member.

37. The device of claim 36, wherein the tissue-piercing member is a needle.

38. The device of claim 35, wherein the first and second curved surfaces, when in the first collapsed position, are configured to substantially surround a vessel and to position at least a portion of a wall of the vessel for piercing by a tissue-piercing member passing through the lumen.

39. A method for forming a tract in tissue using a device comprising first and second curved surfaces that are opposed to each other and coupled at an attachment point, the first and second curved surfaces configured to move about the attachment point between a first collapsed position and a second outwardly displaced position, the method comprising:
moving the first and second curved surfaces from the second outwardly displaced position to the first collapsed position to clamp tissue between the first and second curved surfaces,
wherein the first and second curved surfaces position at least a portion of the clamped tissue for piercing by a tissue-piercing member passing through a lumen defined by the device.

40. The method of claim 39, further comprising advancing a tissue-piercing member through at least a portion of the clamped tissue.

41. A method for forming a tract in tissue of a subject comprising:
clamping at least a portion of tissue; and
advancing a tissue-piercing member in a first direction through at least a portion of the clamped tissue to form a tract in the tissue,
wherein formation of the tract requires advancement of only one tissue-piercing member through the tissue, and wherein the tract is self-sealing.

42. The method of claim 41, wherein the tissue-piercing member comprises a needle.

43. The method of claim 42, wherein the needle is hollow.

44. The method of claim 41, wherein the tissue is clamped between first and second clamping portions of a clamping member.

45. The method of claim 44, wherein the first and second clamping portions comprise first and second elongated clamping arms.

46. The method of claim 44, wherein the clamping member has a first position and a second position, and wherein the first and second clamping portions are farther apart from each other in the first position than they are in the second position.

47. The method of claim 46, further comprising advancing the clamping member to the portion of tissue while the clamping member is in the second position.

48. The method of claim 47, wherein the clamping member is in the first position prior to clamping at least a portion of the tissue.

49. The method of claim 41, wherein the tissue is clamped between opposed first and second curved surfaces coupled at an attachment point and configured to move about the attachment point between a first collapsed position and a second outwardly displaced position.

50. The method of claim 41, further comprising advancing the tissue-piercing member in a second direction through at least a portion of the clamped tissue.

51. The method of claim 41, wherein the tissue comprises a vessel.

52. The method of claim 51, wherein the tissue comprises an artery, and the tract is an arteriotomy.

53. The method of claim 41, further comprising withdrawing the first tissue-piercing member from the tissue, wherein the tract seals after the first tissue-piercing member has been withdrawn.

54. The method of claim 53, wherein the tract seals within 15 minutes or less.

55. The method of claim 53, wherein the tract seals within 5 minutes or less.

56. The method of claim 53, wherein the tract seals within 1 minute or less.

57. The method of claim 41, further comprising advancing one or more tools through the tract.

58. A method for forming a tract in tissue of a subject comprising:
clamping at least a portion of tissue; and
advancing a tissue-piercing member in a first direction through at least a portion of the clamped tissue to form a single tract in the tissue,
wherein the single tract is self-sealing.

59. The method of claim 58, wherein the tissue comprises a vessel.

60. The method of claim 59, wherein the tissue comprises an artery, and the tract is an arteriotomy.

61. The method of claim 58, further comprising withdrawing the first tissue-piercing member from the tissue, wherein the tract seals after the first tissue-piercing member has been withdrawn.

62. The method of claim 61, wherein the tract seals within 15 minutes or less.

63. The method of claim 61, wherein the tract seals within 5 minutes or less.

64. The method of claim 61, wherein the tract seals within 1 minute or less.

65. A method for forming a tract in tissue of a subject comprising:
using a device to clamp at least a portion of tissue; and
forming a tract in the tissue by advancing at least one tissue-piercing member through at least a portion of the clamped tissue,
wherein formation of the tract requires advancement only of the tissue-piercing member through the tissue, and wherein the tract is self-sealing.

66. The method of claim 65, wherein the tissue comprises a vessel.

67. The method of claim 66, wherein the tissue comprises an artery, and the tract is an arteriotomy.

68. The method of claim 65, further comprising withdrawing the first tissue-piercing member from the tissue, wherein the tract seals after the first tissue-piercing member has been withdrawn.

69. The method of claim 68, wherein the tract seals within 15 minutes or less.

70. The method of claim 68, wherein the tract seals within 5 minutes or less.

71. The method of claim 68, wherein the tract seals within 1 minute or less.
